(19) United States
(12) Patent Application Publication
(10) Pub. No.: US 2016/0058689 A1
(43) Pub. Date: Mar. 3, 2016

(54) Compositions for Cosmetic Formulation Comprising A Mixture Selected From Murumuru Butter, Ucuúba Butter, Brazilian-Nut Oil, Passion Fruit Oil, Cupuassu Butter, Assaí Oil and / or Nhandiroba Oil and / or Esters Therefor, As Well As The Use Of A Mixture for Preparation Of A Cosmetic Product

(71) Applicant: Natura Cosméticos S.A., Itapecerica da Serra (BR)

(72) Inventors: Joyce Kazue Alves WADA, Itapecerica da Serra (BR); Marcos Rogerio Spina, Itapecerica da Serra (BR)

(21) Appl. No.: 14/471,770

(22) Filed: Aug. 28, 2014

(51) Int. Cl. A61K 8/92; A61Q 17/00; A61Q 3/02; A61Q 5/00; A61K 8/37; A61Q 19/00

(52) U.S. Cl. CPC A61K 8/922; A61K 8/37; A61Q 19/00; A61Q 3/02; A61Q 5/00; A61Q 17/00; A61K 2800/5922

(57) ABSTRACT

The present invention relates to compositions for cosmetic formulation, consisting of a mixture of components selected from murumuru butter, ucuuba butter, Brazilian-nut oil, passion fruit oil, cupuassu butter, assai oil and nhandiroba oil and/or esters thereof, by means of a mixture for application to the skin, hair, hands and nails. Besides, the invention refers to the use of such a mixture for the preparation of cosmetic products that are technologically differentiated for exhibiting proven efficacy in cosmetics.

### FIELD OF THE INVENTION

The present invention relates to compositions for cosmetic formulation, consisting of a mixture of components selected from murumuru butter, ucuuba butter, Brazilian-nut oil, passion fruit oil, cupuassu butter, assai oil and nhandiroba oil and/or esters thereof, by means of a mixture for application to the skin, hair, hands and nails. Besides, the invention refers to the use of such a mixture for the preparation of cosmetic products that are technologically differentiated for exhibiting proven efficacy in cosmetics, as will be demonstrated in the present patent application.

### BACKGROUND OF THE INVENTION

The skin, which is the largest organ of the human body, consists of a cutaneous barrier responsible for various functions, among which are the protection function, the coating function, the sensorial function, the heat regulation function, among others. The stratum corneum or horny layer, the main barrier of the skin, is formed by cells that are organized in a stacked manner and that are called corneocytes, which are fitted together by a matrix of complex lipids. FIG. 1 illustrates a scheme of the stratum corneum.

The lipids that constitute the stratum corneum are composed by ceramides (50%), cholesterol (25%) and free fatty acids (10%), and also by minor amounts of other esters and sulfates of cholesterol.

The reduction of ceramides in the intercellular lamellae may cause the skin to become very dry. Thus, it is desirable to provide this lipid reduction of ceramides by using cosmetic products of topical application. The ceramides are extremely insoluble compounds, a property directly linked to their intrinsic functionality, that is, the formation of an impermeable layer in the skin.

The stratum corneum contains about 10 to 20% water, and its hydration/moisturizing degree results from the balance between supplied water (either endogenous or exogenous) and the losses by evaporation.

The hydrolipidic film of the skin surface, an emulsion formed by the cutaneous tallow, sweat and components thereof, plays an important role in retaining water. The main factor responsible for drying, scaling and, in more serious cases, dermatitis, may be related to the loss of water of this hydrolipidic film, called transepidermal water loss (TEWL).

Various factors can cause TEWL, as for example, environmental conditions such as cold, wind and low humidity. Other external factors can also attack the cutaneous barrier, removing the natural moisturizing of the skin. These factors include solvents, detergents, excess use of water and toilet soap, among other chemical products. The seriousness of the damages is dependent upon the type and intensity of exposure to these factors.

Skin cleaning products are considered light irritants. These have surfactants for removing dirty, bacteria, fats, perspiration, etc. The repetitive and prolonged exposure to these products result in denaturation of hygroscopic molecules, free amino acids and extracellular substances, such as lipids of the lamellas responsible for cohesion of the stratum and natural hydration/moisturizing.

A dried-up skin loses its biomechanical, biological and, above all, esthetic properties, since its appearance becomes opaque, rough, without elasticity and with a tendency to scaling.

Another part of the human body that is the target of cosmetic treatments is the nail. The nail is a cutaneous attachment that overlaps the back face of the distal phalanges. In the usual language one defines a nail as being the hard plate that is located in the back region of the tip of each finger, growing from about 2 to 4.5 mm a month, or 0.5 to 1.2 mm a week.

The main function of the nails is to protect the distal end of the fingers against traumatism. They have also the function nippers, participate in the discriminating function and are used to scratch. One may not forget also the cosmetic function, which is more important in our environment among women, but which is progressively becoming very important among men as well, within the esthetics, and takes quite a long time for caring.

The nail apparatus is firmly adhered to the periosteum of the distal phalange by dense collagen fibers. Due to its embryonic formation from the primitive epidermis, it has great similarity to a hair and the stratum corneum in both normal and pathologic conditions.

The components of the nail unit, illustrated in FIG. 2, are basically six:
the nail matrix, which is the generating portion;
the nail plate, product of keratinization of the matrix (the nail proper);
the cuticle system, embracing the eponychium or visible cuticle, derived from the proximal nail fold, and the hyponychium, derived from the emedullaelium of the nail bed;
the support portion represented by the nail bed and the bone phalange;
the anchorage portion represented by the specialized mesenchyme, which exists proximally between the phalange and the matrix, and distally between the phalange and the lateral and distal digital pulp;
the skeleton composed by the nail plates: proximal, lateral and distal.

At the distal margin of the nail bed, there is a transverse 1-1.5 mm-strip that represents the maximum linking point of the stratum corneum of the bed and the nail plate. This represents the first and greatest barrier to passage of materials and organisms under the nail plate.

The nail plate (the nail) differs from the skin, because it does not scale off, and from the hair because it does not have cyclic activity. Its flexibility is due to the presence of phospholipids and, on the other hand, the hardness is due to the high content of sulfur.

Another not less important part of the body, also important in aesthetics, are the hairs growing on the scalp, which have the same structure of all hairs of the human body, but with their particularities. The hair is a keratinized strand that grows on the skin of mammals. The hair shaft is the part of the strand that emerges from the scalp and can be divided into three parts:
cuticle: the outer layer of the hair strand that is divided into 5 to 12 layers that, when overlapped, protect the structures. Since they are transparent, one can see the color of the hair. The cuticle undergoes external attacks (sunshine, rain, pollution, etc.) by mechanical action (brushing, combing, etc.) and chemical transformations (relaxing, permanent wave, coloring, highlights, etc.). The cuticles overlap one another partially and may form five to ten layers of plates. These plates, in turn, provide excellent protection to the cortex;
cortex: intermediate region where we transform, in all forms, the hair structure. It represents the heart of the hair. The degree of strength, elasticity and color of the hair depends on its structure. The diameter of the cortex is determined as a function of the number of cells present in the bulb that can multiply. The hair fiber has 2 or 3 types of cortex cells; and
the medulla: central part of the hair. The medulla canal may be empty or filled with spongy keratin. The function of this region has not been determined yet. However, recent studies indicate researches for association of the medulla with the first moment of the hair germination phase, wherein the medulla would serve as a “guide” of the new hair toward the pore.

In this regard, the search for components of renewable sources for consumption, also in the cosmetic area, proves to be important and potential, since the preservation of the environment is one of the factors of the utmost importance at present.

The Amazon region has numberless species of oleaginous plants that exhibit a promising potential in the cosmetic area. Murumuru butter, ucuuba butter, Brazilian-nut oil, passion fruit oil, cupuassu butter, assai oil and nhandiroba oil and/or esters thereof, such as myristyl cupuassuate, are examples of species in this region that exhibit said potential.

Murumuru butter (Astrocaryum murumuru) has a cosmetic action in cleaning and treating hair, inasmuch as it is highly nutritional, emollient and moisturizing, enabling the recovery of moisture and natural elasticity of keratin of hair, skin and nails.

Ucuuba butter (Virola surinamensis) has the property of penetrating the deepest layers of the skin, promoting regeneration of the skin tissues, because it has antiseptic, anti-inflammatory, antiparasitic, emollient, healing and revitalizing properties, by virtue of the cell renewing power of its phytoactives. They also exhibit action on hair and nails.

Brazilian-nut oil (Bertholletia excelsa) has cosmetic action in cleaning and treating hair, inasmuch as it has a high moisturizing power, good formation of foam and refreshing odor, in addition to exhibiting action on skin and nails.

Passion fruit oil (Passiflora edulis/Passiflora incarnata) has cosmetic action in cleaning and treating hair, inasmuch as it provides rest and smoothness to the fibers, besides contributing to restoration of the lipid layer of the skin, providing emollience and softness. It further has extremely reassuring fragrance. Passion fruit oil also has effects on skin and nails.

Cupuassu butter (Theobroma grandiflorum) provides a silky sensorial effect, provides retention of moisture and is composed by fatty acids that aid in recovering the skin, hair and nails.

Assai oil (Euterpe oleracea) has nutritional and protective properties and is indicated for use in hair-care products, chiefly in formulas for nutrition, cleaning and protection of weakened hair, as well as cosmetic effects on skin and nails.

Nhandiroba oil (Fevillea trilobata), in spite of being used as a source for the production of biodiesel, it also has been researched for cosmetic application.

However, prior-art documents related to the cosmetic area of the present invention make clear that the results of the mixtures described in the present patent application are neither suggested nor indicated.

Document EP2026746 describes a method for tanning the skin or coloring the keratin fibers, composition and process for preparation thereof. The composition described has, among others, non-volatile oils and fatty substances and lipophilic compounds that may be, among many, selected from murumuru butter, Brazilian-nut oil (Bertholletia excelsa) and Virola Sebifera (a species of ucuuba). However, this document does not describe or even suggest a mixture consisting specifically of these 3 components in conjunction, nor does it even describe, mention or suggest specifically the effects achieved by effectively using the mixture of murumuru butter, Brazilian-nut oil and ucuuba, as the present invention does.

Document EP2099530 describes improved formulations of oil-soluble UV organic absorbers. In addition to the absorbers established therein, these formulations also comprise dimethyl capramide, a carrier and water. In the composition described, dimethyl caparmide may have solubilizing emollients added, which may be selected, among many, from murumuru butter, Bertholletia excelsa (Brazilian nut) and Virola Sebifera (a species of ucuuba). However, one does not specifically describe, mention or suggest the effects obtained by effectively using the mixture of murumuru butter, Brazilian-nut oil and ucuuba, as the present invention does.

Document EP2170250 describes the use of a nanodispersion comprising a film forming molecule, an emulsifier, a lipophilic component and an oxidation-sensitive, water-soluble ingredient for use in formulations for cosmetic purposes. EP2170250 describes a few oils having emollient properties and cites their capability of retaining in the skin or stratum corneum and, from a vast list, cites murumuru matter, Bertholletia excelsa oil (Brazilian nut) and Virola serbifera (a species of ucuuba). These oils are present in a range of from 0.1 to 80% by weight, based on the other components of the nanodispersion. However, the form and the effects achieved in the present invention are not describes or suggested therein.

Document BRPI1103185 belonging to the present applicant describes cosmetic compositions comprising at least one emollient consisting of oils or fatty-chain plant butters at a concentration of about 0.1% to about 3%. However, specific mixtures of such oils and plant butters and the effects obtained in the present invention are not described or even suggested therein.

It remains the need for the development of cosmetic formulations comprising components of renewable sources that have positive effects and exhibit improved benefits in, for instance, moisturizing, film forming, strengthening the skin barrier, substantiveness and power for restructuring hair and strengthening nails. Particularly, among the improved benefits of the present invention are: differentiated formation of film, such as formation of a protective film; formation of a protective layer; protection against external agents; formation of a protective film; differentiated formation of skin barrier; differentiated moisturizing; substantiveness and power to restructure hair, as for instance, recovery, repair, restructuring and nutrition of hair; reduction of damage to the hair; protection against chemical damages; sealing hair cuticles; penetration into the hair fiber; and differentiated nail strengthening.

### SUMMARY OF THE INVENTION

The present invention relates to compositions for cosmetic formulation comprising a mixture of up to three 3 components selected from murumuru butter, ucuuba butter, Brazilian-nut oil, passion fruit oil, cupuassu butter, assai oil and/or nhandiroba oil and/or the esters thereof.

One of the esters used in the present invention is myristyl cupuassuate.

The present invention further relates to the use of a mixture of up to three components selected from murumuru butter, ucuuba butter, Brazilian-nut oil, passion fruit oil, cupuassu butter, assai oil and/or nhandiroba oil and/or the esters thereof for the preparation of a cosmetic product with a number of benefits, such as moisturizing, formation of film, strengthening the skin barrier, substantiveness and power to restructure hair and strengthen nails.

One of the esters used in the present invention is myristyl cupuassuate.

### BRIEF DESCRIPTION OF THE FIGURES

FIG. 1 refers to the scheme of the stratum corneum illustrating the corneocytes and lipidic layers between them.

FIG. 2 refers to the anatomy of the human nail, which shows the components of the nail unit.

FIG. 3 refers to the average values of bending strength, measured in Force (N) of the Vitro-Nail® slides, before (initial) and after application of the cosmetic treatment (final) for each study group. Mean±SD. (TEST 1.1)

FIG. 4 refers to the results of the complete statistical analysis carried out for the TEST 1.1.

FIG. 5 refers to average values of bending strength, measured in Force (N) of the Vitro-Nail® plates, before (initial) and after application of the cosmetic treatment (final) for each study group. Average±SD (TEST 1.2)

FIG. 6 refers to the results of the complete statistical analysis carried out for TEST 1.2.

FIG. 7 refers to the averages of the variation of the transepidermal loss of water after mechanical insult (Average±SE, n=26) in the beginning of the study and after 14 or 28 days of use of the product with respect to the control (MIXTURE 1)—TEST 2.

FIG. 8 refers to the statistical analysis of the homogeneity of the initial values of TEWL (MIXTURE 1)—TEST 2.

FIG. 9 refers to the statistical analysis of the significance of the variations of transepidermal loss of water throughout the study (MIXTURE 1)—TEST 2.

FIG. 10 refers to the statistical analysis of the significance of the effect of the product with respect to the control (MIXTURE 1)—TEST 3.

FIG. 11 refers to the averages of the variation of the transepidermal loss of water after mechanical insult (Average±, n=26) in the beginning of the study and after 14 or 28 days of use of the product with respect to the control (MIXTURE 2)—TEST 3.

FIG. 12 refers to the statistical analysis of the significance of the variations of transepidermal loss of water through the study (MIXTURE 2)—TEST 2.

FIG. 13 refers to the statistical analysis of the significance of the effect of the product with respect to the control (MIXTURE 2)—TEST 3.

FIG. 14 refers to the averages of the variations of the transepidermal loss of water after mechanical insult (Average±SE, =28) in the beginning of the study and after 14 days of use of the product with respect to the control (MIXTURE 3)—TEST 4.

FIG. 15 refers to the statistical analysis of the homogeneity of the initial values of TEWL (MIXTURE 3)—TEST 4.

FIG. 16 refers to the statistical analysis of the significance of the variations of transepidermal loss of water through the study (MIXTURE 3)—TEST 4.

FIG. 17 refers to the statistical analysis of the significance of the effect of the product with respect to the control (MIXTURE 3)—TEST 4.

FIG. 18 refers to the optical microscopy images representative of the study groups—TEST 5.

FIG. 19 refers to the optical microscopy images representative of the study groups—TEST 5.

FIG. 20 refers to the statistical analysis carried out for the TEST 5.

FIG. 21 refers to the average values of bending strength, measured in Force (N) of the Vitro-Nails® plates, before (initial) and after application of the cosmetic treatment (final) for each study group. Average±SD for the TEST 6.

FIG. 22 refers to the statistical analysis carried out for the TEST 6.

FIG. 23 refers to the complete results of the comparison between the treatments, which was carried using the single-factor variance analysis method, with Tukey post-test, considering a confidence interval of 95% for the TEST 6.

FIG. 24 refers to the illustration of the process of detecting fragments and edges through analysis of images for the TEST 7.

FIG. 25 refers to the results of percentage damages (lifted cuticles, fragments) detected on the hair surface from the analysis of the MEV images. Average±SD—TEST 7.

FIG. 26 refers to the statistical analysis of the results achieved for the groups T01, T02 and T03, which were analyzed through the single-factor variance analysis method, with Tukey multiple comparison post-test, considering a confidence interval of 95% in the TEST 7.

FIG. 27 refers to examples of fluorescence microscopy images of the longitudinal segments representative of the study groups in the TEST 8.

FIG. 28 refers to the fluorescence intensity of the longitudinal segments of the hair fibers for the treatment groups. Average±SD in the TEST 8

FIG. 29 refers to the complete statistical analyses carried out for the TEST 8.

FIG. 30 refers to examples of fluorescence microscopy images of the sectional cuts, representative of the study groups in the TEST 8.

FIG. 31 refers to the intensity of fluorescence of the sectional cuts of the hair fibers for the treatment groups. Average±standard deviation in the TEST 8.

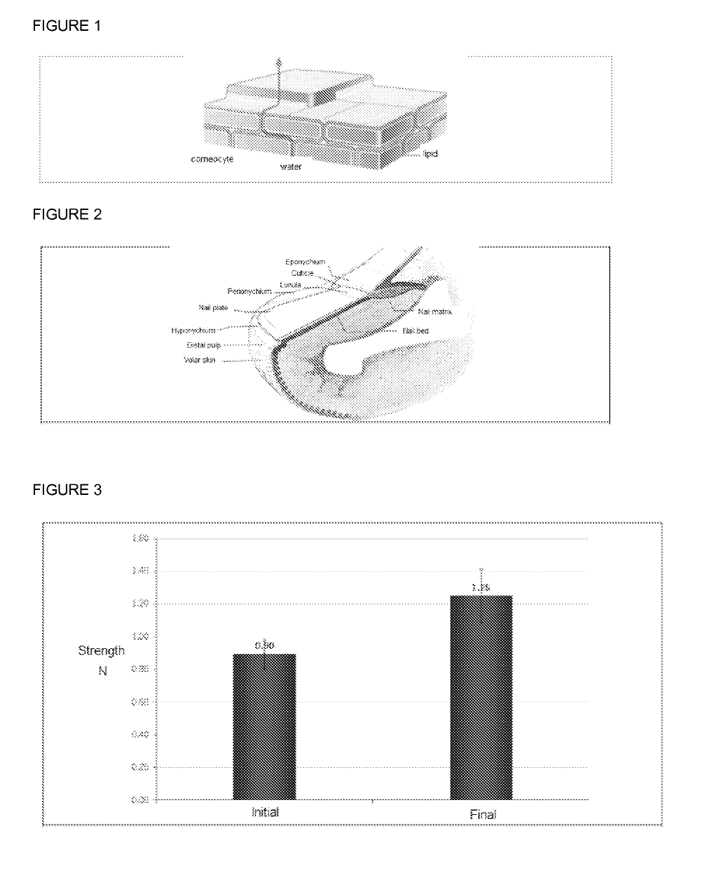

### DETAILED DESCRIPTION OF THE INVENTION

The present invention relates to compositions for cosmetic formulation, comprising a mixture of up to 3 components selected from murumuru butter, ucuuba butter, Brazilian-nut oil, passion fruit oil, cupuassu butter, assai oil and nhandiroba oil and/or esters thereof, such as myristyl cupuassuate. Moreover, commercially acceptable adjuvants, directed to application in the cosmetic, hygiene and personal-care industry are applied.

It was found that mixtures of these specific components provide improved results. The presently described mixtures proved to be effective in the treatment of the skin, since they promoted film formation, barrier strengthening and immediate moisturizing, as well as enhanced nail strengthening. Particularly, among the improved benefits of the present invention are: differentiated formation of film, such as formation of a protective film; formation of a protective layer; protection against external agents; formation of a protective film; differentiated formation of skin barrier; differentiated moisturizing; substantiveness and power to restructure hair, as for instance, recovery, repair, restructuring and nutrition of hair; reduction of damage to the hair; protection against chemical damages; sealing hair cuticles; penetration into the hair fiber; and differentiated nail strengthening. Preferably, the invention relates to compositions comprising the following constitutions:

TABLE A

| | Concentration (% by | |
|---|---|---|
| Component | weight) | Function |
| Murumuru butter | 0 to 40 | Emollient |
| Ucuuba seed butter | 0 to 40 | Emollient |
| Brazilian-nut oil | 0 to 60 | Emollient |
| Commercially | qsp | Carrier |
| acceptable adjuvants | | |

The composition A comprises from 0 to 40% by weigh murumuru butter (emollient), from 0 to 40% by weigh ucuuba seed butter (emollient), from 0 to 60% by weigh Brazilian-nut oil (emollient) and commercially available adjuvants in an amount cosmetically acceptable in the composition.

TABLE B

| | Concentration (% by | |
|---|---|---|
| Component | weight) | Function |
| Passion fruit seed | 0 to 70 | Emollient |
| butter | | |
| Cupuassu seed butter | 0 to 50 | Emollient |
| Myristyl cupuassuate | 0 to 30 | Emollient |
| fatty ester | | |
| Cosmetically | qsp | Carrier |
| acceptable adjuvants | | |

The composition A comprises from 0 to 70% by weigh passion fruit seed butter (emollient), from 0 to 50% by weigh cupuassu seed butter (emollient), from 0 to 30% by weigh myristyl cupuassuate fatty ester (emollient) and commercially available adjuvants in an amount cosmetically acceptable in the composition.

TABLE C

| | Concentration (% by | |
|---|---|---|
| Component | weight) | Function |
| Assai oil | 1 to 30 | Emollient |
| Nhandiroba oil | 70 to 99 | Emollient |
| Cosmetically | qsp | Carrier |
| acceptable adjuvants | | |

The composition C comprises 1 to 30% by weight assai oil (emollient), 70 to 99% by weight nhandiroba oil (emollient), and cosmetically acceptable adjuvants in an amount cosmetically acceptable in the composition.

The cosmetic composition of the present invention has a number of advantages and desired characteristics in a cosmetic product, particularly for the skin, hair, hands and nails, these advantages being achieved with the ideal and balanced combination between its components, such as:
differentiated formation of film, such as formation of protective film; formation of a protective layer; protection against external agents; formation of protective film;
strengthening of the differentiated skin barrier;
differentiated moisturizing;
differentiated substantiveness and power to restructure the hair, such as recovery, repair, restoration and nutrition of the hair; reduction of damage to the hair; protection against chemical damage; sealing of hair cuticles; penetration into the hair fiber; and
differentiated strengthening of the nails.

In a non-exclusive manner, the cosmetic compositions of the present invention can be advantageously used for preparing cosmetic products such as elixirs, emulsions for body and hands, emulsions for the face, anhydrous formulas for body and hands, anhydrous formulas for the face, emulsions for the hair, anhydrous formulas for the hair. These compositions can be included in all types of formulations.

The exemplified embodiments of the invention below are intended for illustration of the invention, without any limitation of the scope thereof.

### Examples

### Example Mixture 1

### Cosmetic Composition 1 of the Present Invention

Table 1 below presents a formulation of the cosmetic composition of the present invention.

MIXTURE 1

| Component | Concentration (% by weight) | Function |
|---|---|---|
| Murumuru butter | 25% | Emollient |
| Ucuuba seed butter | 25% | Emollient |
| Brazilian-nut oil | 50% | Emollient |

The mixture 1 comprises 25% by weigh murumuru butter (emollient), 25% by weight ucuuba seed butter (emollient) and 50% by weight Brazilian-nut oil (emollient).

### Example Mixture 2

### Cosmetic Composition 2 of the Present Invention

Table 2 below presents a formulation of the cosmetic composition according to the present invention.

MIXTURE 2

| | Concentration | |
|---|---|---|
| Component | (% by weight) | Emollient |
| Passion fruit seed oil | 50% | Emollient |
| Cupuassu seed butter | 35% | Emollient |
| Myristyl cupuassuate fatty ester | 15% | Emollient |

The mixture 2 comprises 50% by weight of passion fruit seed oil (emollient), 35% by weight cupuassu seed butter (emollient) and 15% by weight myristyl cupuassuate fatty ester (emollient).

### Example Mixture 3

### Cosmetic Composition 3 of the Present Invention

Table 3 below presents a formulation of the composition according to the present invention.

MIXTURE 3

| Component | Concentration (% by weight) | Function |
|---|---|---|
| Assai oil | 10% | Emollient |
| Nhandiroba oil | 90% | Emollient |

The mixture 3 comprises 10% by weight assai oil (emollient) and 90% by weight nhandiroba oil (emollient).

### Tests:

The cosmetic composition (MIXTURE 1, MIXTURE 2 and MIXTURE 3) cited and defined in the above examples were the compositions applied in the tests described hereinafter.

On the other hand, the parameter used as “control” for the purposes of comparison with the mixtures of the present invention is an area of the skin without application of any product onto it. As far as the hair is concerned, the control refers to a lock of hair duly washed, wherein all the locks have been subjected to a standardized pre-cleansing process. In turn, in the film formation test (TEST 5), the controls are substrates hydrated with distilled water.

The expression “phototype” used in these tests hereinafter is a Fitzpatrick classification based on the relationship of sunburn on six types of skin:

Phototype I—white skin, very sensitive to sunshine. The skin burns very easily and never becomes tanned;

Phototype II—white skin, very sensitive to sunshine. The skin burns easily and becomes tanned very little;

Phototype III—cream white, which has normal sensitivity to sunshine. The skin burns and becomes tanned moderately;

Phototype IV—Moderate brown skin, normal sensitive to sunshine. The skin burns a little and becomes tanned moderately;

Phototype V—dark brown skin, little sensitive to sunshine. The skin rarely burns and becomes much tanned;

Phototype VI—black skin, which is insensitive to sunshine. It never burns and is totally pigmented.

### TEST 1—Comparative Test

1. Physicochemical Characterization of the Sample Tested:

The mixture tested presented a composition rich in meddle-chain acids: 17% lauric acid, 24% myristic acid, besides 10% palmitic acid, 19% oleic acid and 21% linoleic acid. The triacylglyceridic composition, by chain size, exhibited a high concentration of groups C42 (19%), C44 (18%) and C40 (12%), due to the high concentration of fatty acids C14, C12 and C16. Besides, the groups C52 and C54 exhibited important concentrations: 14 and 13%, respectively, due to the presence of oleic and linoleic acids. Many physical properties of oils and butters such as crystalline structure, viscosity and melting point are influenced by the structures of these triacylglycerols.

The chemical structure of this mixture resulted in an interesting solid profile behavior: 42% solids at 10° C. and only 1% at 35° C., that is to say, solid active at cooling temperature, becoming liquid at the skin temperature.

Table 4 compares physic chemically the composition in fatty acid of mixtures 1, 2 and 3:

TABLE 4
Physicochemical comparison of the composition in fatty acid
of mixtures 1, 2 and 3

| | Mixture 1 (%) | Mixture (2) | Mixture 3 (%) |
|---|---|---|---|
| C12: 0 lauric | 16.9 | 0 | 0 |
| C14: 0 myristic | 24.1 | 0 | 0.2 |
| C16: 0 palmitic | 10.1 | 7.8 | 30.8 |
| C16: 1 palmitoleic | 0.3 | 0 | 0.1 |
| C18: 0 stearic | 7.2 | 14 | 10.3 |
| C18: 1 oleic | 18.6 | 21.8 | 14.7 |
| C18: 2 linoleic | 21.4 | 35.7 | 7.4 |
| C18: 3 linoleic | 0.1 | 0.2 | 0.1 |
| C20: 0 arachidic | 0.2 | 4.2 | 0.3 |
| C20: 1 eicosanoic | 0 | 0.1 | 0.1 |
| C22: 0 bohenic | 0 | 0.7 | 0 |
| C24: 0 lignoceric | 0 | 0.1 | 0.1 |
| Conjugated | 0 | 0 | 35 |
| dienesand trienes | | | |
| Myristyl esters | | 15% | |

1.2 Proof of Efficacy

The mixture promoted moisturizing of the skin, evidenced by significant alterations of corneometry, at significance level of 5%, in the times 15 min, 4 h, 6 h and 8 h with respect to the control.

It was also found that the application of the sample onto the artificial skin provided formation of film significantly superior to the control. Thus, one can conclude that the mixture is capable of forming film on the human skin.

The mixture applied to the skin in the region of the forearm imparted significant strengthening of the skin barrier as compared to the control after 14 and 28 days of continuous use. The percentage values obtained for the strengthening of the skin barrier with respect to the initial state of the skin were of 14% after 14 days of use and 21% after 28 days.

According to the results of the stress-deformation assay, the application of the mixture onto synthetic nails increased by 76% the force for bending thereof. Thus, it was proven that the treatment with the mixture promoted a significant strengthening of the nails.

The application of the moisturizer containing 1% elixir increased by 40% the force necessary to bend the nails; on the other hand, the product containing 1% D-Pantenol increased by 16% the force to bend the nails.

2. Discussion and Results

It was found that the proposed mixture:
proved to be effective in the treatment of the skin, since it promoted the formation of film, strengthening the barrier and immediate moisturizing for up to 8 h;
promoted increase by 76% of nail strengthening.

The presently described mixtures proved to be effective in the treatment of the skin, since it promoted film formation, barrier strengthening and immediate moisturizing; it further promoted increase in the strengthening of the nails, as well application of the mixture at 1% in emulsion exhibited a result superior to another active on the market, evaluated at the same concentration.

The mixture that contains murumuru butter, ucuuba and Brazilian-nut oil is an effective ingredient from the Amazon region for application in cosmetic products.

According to the results achieved of the stress-deformation essay, the application of the product Emulsion Hands containing 1% of M1 onto the synthetic nails increased by 40% (or 1.4 times) the force necessary to bend them.

According to the results achieved of the stress-deformation essay, the application of the product Moisturizer Hands containing 1% or market active onto the synthetic nails increased by 16% (or 1.2 time) the force necessary to bend them.

These results are described in detail hereinafter.

### TEST 1.1—Comparative Test 1.1

### 1. Experimental Design

The nail strengthening study was carried out in-vitro on a synthetic nail mimicking the human nail, Vitro-Nails®, manufactured by IMS-USA.

Vitro-Nails® contains lipidic and protein components, mimicking the wetting, thickness and flexibility properties of human nails (Sottery, J. P.; Jaramillo, J. H. A New Substrate for the Rapid, In Vitro Assessment of Nail Care Products. IMS Inc, Society of Cosmetic Chemists Annual Scientific Metting, 1998).

Ten Vitro-Nails® synthetic nail plates underwent cosmetic treatment with the product known as “Moisturizer Hands” whose formulation is described below.

TABLE 5
Formulation of “Moisturizer Hands”

| Description | (%) |
|---|---|
| Aluminum starch Octenylsuccinate | 1 |
| AQUA (or) water | 78.95 |
| ARISTOFLEX AVL | 0.5 |
| ELIXIR | 1 |
| butyl-hydroxytoluene (BHT) | 0.05 |
| Caprylic/capric triglyceride | 1 |
| Ceteareth-20 | 1.5 |
| Cetearyl alcohol | 3.5 |
| Dimethicone | 0.5 |
| Disodium EDTA | 0.1 |
| DMDM Hydantoin | 0.6 |
| Glycerin | 5 |
| Glyceryl stearate and PEG-100 stearate | 2 |
| Glyceryl stearate/Glyceryl distearate | 2 |
| Methylisothiazolinone | 0.1 |
| Myristyl THEOBROMA GRANDIFLORUM SEEDATE | 1.5 |
| Propylheptyl caprylate | 0.5 |
| Xanthan gum (Keltrol SFT) | 0.2 |
| | 100 |

The plates underwent a pre-cleansing procedure in which a paper towel soaked with a nail polish remover was used.

After cleansing, basal (Initial) measurements of the bending strength of the plates were obtained with the use of a universal testing machine EMIC DL500, with a 20-N load cell.

After obtaining the basal measurements, 50 μl of the product were applied onto each plate. The product was spread all over the surface (of one of the sides of the plate) during 1 minute. After that, the plates were kept in an oven at 36° C. for 15 minutes to dry and new bending strength measurements were taken—final measurements (Final).

2. Bending strength measurements

Nail strengthening is related to the force necessary to bend a nail. From the stress-strain curves obtained from the study, the force values—in Newton (N)—necessary to vertically bend the nails by 1.0 mm were obtained.

For plate bending, a proper support was used having a free horizontal span of 35 mm as well as a universal testing machine EMIC, having a load cell of 20N. Descending speed of the probe tip was 10 mm/min.

### 3. Results and Discussions

The bending strength results for the group of study put to the test are shown below:

TABLE 6
Experimental Data Obtained: Force Values (N) for 1.0 mm deformation
Moisturizer Hands

| Initial | Final |
|---|---|
| 1.32 | 2.28 |
| 0.90 | 1.68 |
| 1.08 | 1.92 |
| 0.78 | 1.32 |
| 0.84 | 1.50 |
| 1.20 | 2.10 |
| 0.72 | 1.20 |
| 0.96 | 1.80 |
| 0.84 | 1.44 |
| 0.78 | 1.38 |

FIG. 3 displays the average results obtained: Average bending strength values in (N) of the Vitro-Nails® plates before (initial) and after cosmetic treatment (final) for each group of study. Average±SD.

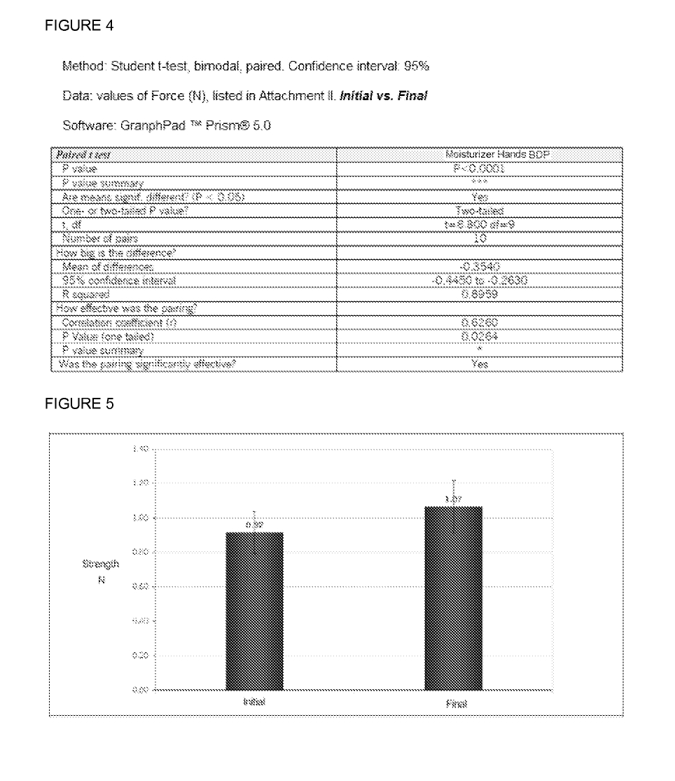

The bending strength data (initial and final ones) were compared on a statistic basis, via the paired, bimodal Student's t-Test method, in which a 95% confidence interval was considered. The results of the complete statistical analysis are shown in FIG. 4.

According to the results, the Vitro-Nails® plates subjected to the application of Moisturizer Hands are more bending-resistant than the Vitro-Nails® plates that did not undergo treatment (initial state).

Table 7 displays the “nail strengthening potential” (PF), in percent values and in number of times calculated in relation to the initial state, according to Equations 1 and 2.

$${P}_{F} ( \% ) = 100 * ( \frac{{F}_{final} - {F}_{initial}}{{F}_{initial}} )$$

Equation 1: Calculation of the nail strengthening potential (%) of the treatment in relation to the initial state, wherein: Fᵢₙᵢₜᵢₐₗ=Force values for the initial state; F_{final}=Force values for the final state.

$${P}_{F} = \frac{{F}_{final}}{{F}_{initial}}$$

Equation 2: Calculation of the nail strengthening potential (in number of times) of the treatment in relation to the initial state, wherein: Fᵢₙᵢₜᵢₐₗ=Force values for the initial state; F_{final}=Force values for the final state.

TABLE 7
Nail strengthening potential after cosmetic treatment in relation to
the initial state (without treatment).

| Treatment | % | Number of times |
|---|---|---|
| Moisturizer Hands whose formulation is | 40 | 1.4 |
| displayed in Table 5 | | |

### 4. Conclusions

In the present study, Vitro-Nails® synthetic nail plates underwent cosmetic treatment with the following product:
Moisturizer Hands whose formulation is displayed in Table 5

According to the results obtained from the stress-strain testing, the application of the product “Moisturizer Hands”, whose formulation is displayed in Table 5, onto the synthetic nails increased by 40% (or 1.4 time) the force necessary to bend the nails.

Thus, we can say that the treatment with Moisturizer Hands, whose formulation is displayed in Table 5, results in significant nail strengthening.

Whereas according to IMS-USA (www.ims-usa.with) the substrate Vitro-Nail® is regarded as a material that mimics the human nail, we infer that the properties obtained from this “in vitro” study can be extrapolated to the human nail.

### TEST 1.2—Comparative Test 1.2

### 1. Experimental Design

The nail strengthening study was carried out in-vitro on a synthetic nail mimicking the human nail, Vitro-Nails®, manufactured by IMS-USA. Vitro-Nails® contains lipidic and protein components, mimicking the wetting, thickness and flexibility properties of human nails (Sottery, J. P.; Jaramillo, J. H. A New Substrate for the Rapid, In Vitro Assessment of Nail Care Products. IMS Inc, Society of Cosmetic Chemists Annual Scientific Metting, 1998).

Ten Vitro-Nails® synthetic nail plates underwent cosmetic treatment with the product known as “Moisturizer Hands”, whose formulation is described below.

TABLE 8
Formulation of “Moisturizer Hands”

| Description | (%) |
|---|---|
| Aluminum starch Octenylsuccinate | 1 |
| AQUA (or) water | 78.95 |
| ARISTOFLEX AVL | 0.5 |
| butyl-hydroxytoluene (BHT) | 0.05 |
| Caprylic/capric triglyceride | 1 |
| Ceteareth-20 | 1.5 |
| Cetearyl alcohol | 3.5 |
| Dimethicone | 0.5 |
| Disodium EDTA | 0.1 |
| DMDM Hydantoin | 0.6 |
| D-PANTHENOL | 1 |
| Glycerin | 5 |
| Glyceryl stearate and PEG-100 stearate | 2 |
| Glyceryl stearate/Glyceryl distearate | 2 |
| Methylisothiazolinone | 0.1 |
| Myristyl THEOBROMA GRANDIFLORUM SEEDATE | 1.5 |
| Propylheptyl caprylate | 0.5 |
| Xanthan gum (Keltrol SFT) | 0.2 |
| | 100 |

The plates underwent a pre-cleansing procedure in which a paper towel soaked with a nail polish remover was used.

After cleansing, basal (Initial) measurements of the bending strength of the plates were obtained with the use of a universal testing machine EMIC DL500, with a 20-N load cell.

After obtaining the basal measurements, 50 μl of the product were applied onto each plate. The product was spread all over the surface (of one of the sides of the plate) during 1 minute. After that, the plates were kept in an oven at 36° C. for 15 minutes to dry and new bending strength measurements were taken—final measurements (Final).

### 2. Bending Strength Measurements

Nail strengthening is related to the force necessary to bend a nail. From the stress-strain curves obtained from the study, the force values—in Newton (N)—necessary to vertically bend the nails by 1.0 mm were obtained.

For plate bending, a proper support was used having a free horizontal span of 35 mm as well as a universal testing machine EMIC, having a load cell of 20N. Descending speed of the probe tip was 10 mm/min.

### 3. Results and Discussions

The bending strength results for the group of study put to the test are shown below:

TABLE 9
Experimental Data Obtained: Force Values (N) for 1.0 mm deformation
Moisturizer Hands

| Initial | Final |
|---|---|
| 0.86 | 0.85 |
| 0.77 | 1.04 |
| 0.89 | 0.98 |
| 0.87 | 0.91 |
| 1.18 | 1.33 |
| 1.00 | 1.24 |
| 0.91 | 1.10 |
| 0.89 | 0.94 |
| 1.02 | 1.09 |
| 0.78 | 1.17 |

FIG. 5 displays the average results obtained: Average bending strength values in (N) of the Vitro-Nails® plates before (initial) and after cosmetic treatment (final) for each group of study. Average±SD.

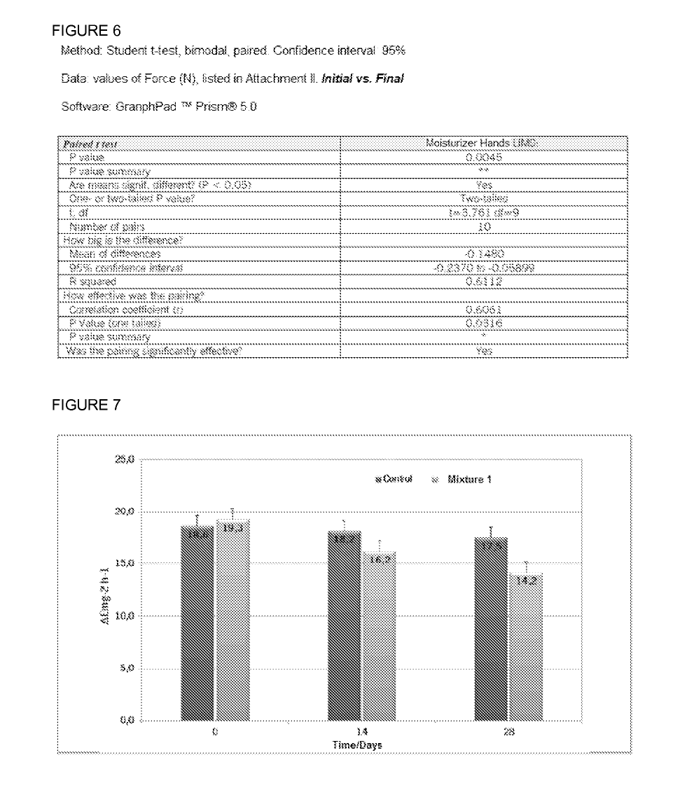

The bending strength data, initial and final ones, were statistically compared by the paired, bimodal Student's t-Test method, considering a confidence interval of 95%. The results of the complete statistical analysis are shown in FIG. 6.

According to the results, the Vitro-Nails® plates subjected to the application of Moisturizer Hands of table 8 are more bending-resistant than the Vitro-Nails® plates that did not undergo treatment (initial state).

Table 10 displays the “nail strengthening potential” (PF), in percent values and in number of times calculated in relation to the initial state, according to Equations 1 and 2.

$${P}_{F} ( \% ) = 100 * ( \frac{{F}_{final} - {F}_{initial}}{{F}_{initial}} )$$

Equation 1: Calculation of the nail strengthening potential (%) of the treatment in relation to the initial state, wherein: Fᵢₙᵢₜᵢₐₗ=Force values for the initial state; F_{final}=Force values for the final state.

$${P}_{F} = \frac{{F}_{final}}{{F}_{initial}}$$

Equation 2: Calculation of the nail strengthening potential (in number of times) of the treatment in relation to the initial state, wherein: Fᵢₙᵢₜᵢₐₗ=Force values for the initial state; F_{final}=Force values for the final state.

TABLE 10
Nail strengthening potential after cosmetic treatment in relation to
the initial state (without treatment).

| Treatment | % | Number of times |
|---|---|---|
| Moisturizer Hands whose formulation is | 16 | 1.2 |
| displayed in Table 8 | | |

### 4. Conclusions

In the present study, Vitro-Nails® synthetic nail plates underwent cosmetic treatment with the following product:
Moisturizer Hands whose formulation is displayed in Table 8

According to the results obtained from the stress-strain testing, the application of the product “Moisturizer Hands”, whose formulation is displayed in Table 8, onto the synthetic nails increased by 16% (or 1.2 time) the force necessary to bend the nails.

Thus, we can say that the treatment with Moisturizer Hands, whose formulation is displayed in Table 8, results in significant nail strengthening.

Whereas according to IMS-USA (www.ims-usa.with) the substrate Vitro-Nail® is regarded as a material that mimics the human nail, we infer that the properties obtained from this “in vitro” study can be extrapolated to the human nail.

### TEST 2—Evaluation of the Strengthening of Skin Barrier Caused by the Use of Cosmetic Product

### 1. Objective

To evaluate the strengthening effect of the skin barrier caused by the home use of MIXTURE 1 during 28 days by using a process for removing layers of the stratum corneum with 30 consecutive applications and removals of adhesive tape and evaluate the transepidermal water loss by evaporimetry.

### 2. Table of Volunteers

The volunteers were instructed to discontinue the use of any topical product in the region of the forearms 48 hours prior to the beginning of the study. They also received instructions regarding the times for conducting the trials and were informed not to use any products during the period the study was being carried out other than the provided product.

### 3. Procedures for Conducting Evaluations

3.1. Climatization

Prior to the beginning of the evaluation, the volunteers remained in the laboratory with the forearms exposed and at rest at 20±2° C. and 50±5% relative humidity for at least 30 minutes.

3.2. Measuring area

In each volunteer's anterior surface of the forearm (right and left) an area of 2.5×4.0 cm was marked with the aid of a guide and a surgical pen, and the arm for application of the product and the control arm were ordered alternately, as one can see in Table 11 below.

TABLE 11

| Volunteer | Age | | Area of application | |
|---|---|---|---|---|
| Code | (years) | Phototype | Right forearm | Left forearm |
| 1 | 55 | III | Mixture 1 | Control |
| 2 | 56 | IV | Control | Mixture 1 |
| 3 | 35 | III | Mixture 1 | Control |
| 4 | 28 | IV | Control | Mixture 1 |
| 5 | 47 | IV | Mixture 1 | Control |
| 6 | 36 | IV | Control | Mixture 1 |
| 7 | 51 | III | Mixture 1 | Control |
| 8 | 53 | III | Control | Mixture 1 |
| 9 | 37 | IV | Mixture 1 | Control |
| 10 | 27 | IV | Control | Mixture 1 |
| 11 | 51 | IV | Mixture 1 | Control |
| 22 | 50 | IV | Control | Mixture 1 |
| 23 | 48 | IV | Mixture 1 | Control |
| 24 | 44 | IV | Control | Mixture 1 |
| 25 | 39 | IV | Mixture 1 | Control |
| 26 | 54 | IV | Control | Mixture 1 |
| 28 | 54 | III | Control | Mixture 1 |
| 29 | 45 | IV | Mixture 1 | Control |
| 37 | 47 | III | Control | Mixture 1 |
| 38 | 53 | IV | Mixture 1 | Control |
| 39 | 52 | IV | Control | Mixture 1 |
| 40 | 40 | IV | Mixture 1 | Control |
| 41 | 43 | IV | Control | Mixture 1 |
| 42 | 52 | IV | Mixture 1 | Control |
| 43 | 37 | IV | Control | Mixture 1 |
| 44 | 57 | IV | Mixture 1 | Control |
| 45 | 53 | IV | Control | Mixture 1 |
| 46 | 50 | IV | Mixture 1 | Control |

3.3. Equipment

The measurements were taken with the aid of a Tewameter® 300 probe coupled to the Multi Probe Adapter MPA-5 (CKeletronics, Germany). A basal measurement of each marked area was carried out and another one after removing the layers of stratum corneum with adhesive tape.

3.4. Taking measurements

On the first day of study, D0, the measurement of the transepidermal water loss (E), in g m-2 h-1 of the untreated skin (T0), prior to removing the layers of the stratum corneum in both areas was obtained: E_{T0D0}. Subsequently, the layers of the stratum corneum were removed by sticking on and pulling out the Transpore® 3M adhesive tape (T30) 30 consecutive times followed by the evaluation of the transepidermal water loss: E_{T30D0}.

Observation: on the first day of the study the difference between ΔEp−ΔEc must be no greater than 3.0 mg⁻²h⁻¹, in order to ensure the basal homogeneity between the area where the product will be applied and the control area. In the event said difference is greater than 3.0 mg⁻²h⁻¹, the procedure must be carried out once more and the areas must be marked on other regions of the forearms. In the event said difference is still noticed, the volunteer must be dismissed.

The volunteers were back to the lab after 14 and 28 days of home use of the product, according to the Study Plan, for new assessments of the transepidermal water loss, untreated skin and after the stripping of layers of the stratum corneum: E_{T0Di}, E_{T30Di}.

3.5. Applying the Product

The product was first applied at the laboratory, by the volunteer, after the measurement of transepidermal water loss was taken after the stripping of the layers of the stratum corneum. Afterwards, the volunteers were requested to apply the product.

The volunteers were told not to use any products on the region of the control forearm throughout the period the study was to be conducted.

### 4. Data Analysis and Interpretation

4.1. Software for Obtaining Average Values and Data Analysis:
MPA for Windows® NT/XP (CKeletronic, Germany, 2004).
Microsoft® Office Excel 2003 (Microsoft Corp., USA, 2003).

4.2. Software for Statistical Analysis:
GraphPad™ Prism® 4.03 (GraphPad Software, San Diego Calif. USA, www.graphpad.com).

4.3. Interpreting the results

The strengthening effect of the skin barrier caused by the continuous use of the cosmetic product can be noticed due to the slighter water loss even after the removal of layers of the stratum corneum, which exposes deeper skin layers.

4.3.1. Calculations

From the gross values of TEWL, herein referred to as E, the following parameters were calculated: variation in the transepidermal water loss due to removal of layers of the stratum corneum, ΔE, ratio between the variations obtained during the study, RE, and the variation percentage of the transepidermal water loss, or strengthening of the skin barrier, FB, as shown in Equations 1 to 3.

ΔE_{Di,X}=E_{T30,Di,X}−E_{T0,Di,X}

Equation 1. Variation in the transepidermal water loss after i days of study, i=0, 14 or 28 days. E_{T30Di}=TEWL value after i days of study and 30 stratum corneum layer stripping (T30). E_{T0Di}=TEWL value after i days of study, obtained from the untreated skin (T0); X=control or product.

RE_{Dj,X}=ΔE_{Dj,X}/ΔE_{D0,X}

Equation 2. Calculation of the ratio between variations in transepidermal water loss after j days of study (Dj, j=14 or 28 days) in relation to the initial condition (D0); X=control or product.

FB_{P}=100*(ΔE_{D0,P}−ΔE_{Dj,P})/ΔE_{D0,P}−100*(ΔE_{D0,C}−ΔE_{Dj,C})/ΔE_{D0,C}

Equation 3. Calculation of the percentage value of the strengthening of the skin barrier for the product P in relation to the control, C, after j days of study (j=14 or 28 days).

4.3.2. Statistical Evaluations

4.3.2.1. Basal Homogeneity

The homogeneity of the basal data, necessary to evince that the final results were not influenced by the initial condition, was assessed by applying the paired, bimodal Student's t-Test method, in which a 95% confidence interval was considered, to data on Δ_{ED0,P} vs. ΔED0,C, wherein C=control, P=assessed product. Satisfactory results are achieved when there is no statistically significant difference between the initial conditions (P>0.05), evincing that there was no difference between the two forearms, i.e., between the areas where product and control were assessed.

4.3.2.2. Significance of the Effect

The significance of the changes in the skin barrier evaluated each time the volunteer returns, both for control and product, is assessed by employing the paired, bimodal Student's t-Test method, in which a 95% confidence interval was considered, with the data on ΔE_{Dj,X} vs ΔE_{D0,X}, wherein X=P (product) or C (control) and j=14 or 28 days.

The adequate results are achieved when there is no statistically significant difference between ΔE_{Di} and ΔE_{D0} (P>0.05) when it comes to control and, when it comes to product, ΔE_{Di} is significantly lower to ΔE_{D0} (P<0.05), which indicates reduction of the TEWL in relation to the strengthening of the barrier.

4.3.2.3. Comparison Between Product and Control

The final evaluation of the significance of the observed effect of strengthening of the skin barrier due to the use of the product was carried out by employing paired, bimodal Student's t-Test method (product vs. control), in which a 95% confidence interval was considered, with data on RE_{Dj,P} vs. RE_{Dj,C}.

The adequate results are achieved when the ratio between the variations in the TEWL obtained for product is significantly lower to the one obtained for control: RE_{Dj,P}<RE_{Dj,C} (P<0.05).

### 5. Results and Discussions:

5.1. Statistics on the Participation of Volunteers

Total contacted volunteers: 69;

Total of participant volunteers: 43 (62% of the contacted volunteers);

Total absences in the day of the study: 14;

Total volunteers dismissed after evaluation of inclusion and exclusion criteria: 1;

Effectively included volunteers: 28 (65% of the acceptances);

Volunteers who completed the study: 26 (93%).

5.2. General Data on the Study Group:

Average age: 46±8 years.

5.3. Climate Control

Statistical data on the environmental monitoring throughout the days the study was carried out at the laboratory for evaluation and climatization of the volunteers:

Day 1 (08:00 a.m. to 06:00 p.m.):

Temperature: (20.6±0.4° C. (95% Confidence interval: 20.5° C. to 20.8° C.)

Relative air humidity: (53±1) % (95% Confidence interval: 53% to 54%)

Day 2 (08:00 a.m. to 06:00 p.m.):

Temperature: (20.8±0.3° C. (95% Confidence interval: 20.6° C. to 21.0° C.)

Relative air humidity: (52±2) % (95% Confidence interval: 51% to 53%)

Day 3 (08:00 a.m. to 06:00 p.m.):

Temperature: (21.2±0.4° C. (95% Confidence interval: 21.0° C. to 21.3° C.)

Relative air humidity: (50±2) % (95% Confidence interval: 49% to 51%)

Day 4 (08:00 a.m. to 06:00 p.m.):

Temperature: (20.5±0.4° C. (95% Confidence interval: 20.4° C. to 20.7° C.)

Relative air humidity: (54±1) % (95% Confidence interval: 54% to 55%)

Day 5 (08:00 a.m. to 06:00 p.m.):

Temperature: (19.8±1.1° C. (95% Confidence interval: 19.3° C. to 20.3° C.)

Relative air humidity: (55±4) % (95% Confidence interval: 54% to 57%)

Day 6 (08:00 a.m. to 06:00 p.m.):

Temperature: (20.6±0.6° C. (95% Confidence interval: 20.4° C. to 20.9° C.)

Relative air humidity: (55±4) % (95% Confidence interval: 54% to 57%)

Day 7 (08:00 a.m. to 06:00 p.m.):

Temperature: (21.0±0.4° C. (95% Confidence interval: 20.8° C. to 21.1° C.)

Relative air humidity: (49±3) % (95% Confidence interval: 48% to 50%)

Day 8 (08:00 a.m. to 06:00 p.m.):

Temperature: (21.2±0.6° C. (95% Confidence interval: 20.9° C. to 21.4° C.)

Relative air humidity: (53±3) % (95% Confidence interval: 52% to 54%)

Day 9 (08:00 a.m. to 06:00 p.m.):

Temperature: (21.4±0.7° C. (95% Confidence interval: 21.1° C. to 21.7° C.)

Relative air humidity: (48±3) % (95% Confidence interval: 46% to 49%)

According to the data registered in the climate control, the temperature and humidity in the laboratory for measurements and climatization of the volunteers remained within the range established in the study protocol on all evaluation days.

5.4. Results Obtained from the Evaluation

The raw data obtained from transepidermal water loss are listed below in Tables 12 to 12.4, as well as the calculated parameters: ΔE (Equation 1), RE (Equation 2) and FB (Equation 3).

TABLE 12
Transepidermal water loss values (E, g m⁻² h⁻¹) for MIXTURE 1

| | | | |
|---|---|---|---|
| Volunteer | D0 | D14 | D28 |

| Code | T0 | T30 | T0 | T30 | T0 | T30 |
|---|---|---|---|---|---|---|
| 1 | 11.5 | 31.3 | 14.8 | 30.1 | 15.7 | 29.8 |
| 2 | 10.6 | 25.6 | 11.3 | 30.1 | 11.9 | 28.0 |
| 3 | 10.3 | 32.2 | 9.7 | 36.0 | 11.8 | 28.5 |
| 4 | 10.5 | 34.2 | 10.5 | 30.7 | 15.6 | 35.9 |
| 5 | 14.9 | 28.0 | 18.5 | 33.8 | 19.9 | 34.9 |
| 6 | 12.5 | 30.1 | 18.1 | 33.2 | 16.1 | 31.7 |
| 7 | 11.2 | 30.5 | 9.2 | 28.3 | 9.7 | 28.8 |
| 8 | 13.0 | 31.6 | 10.5 | 29.1 | 9.2 | 26.8 |
| 9 | 13.1 | 38.0 | 14.9 | 37.3 | 15.8 | 32.0 |
| 10 | 8.2 | 19.0 | 9.0 | 19.3 | 12.7 | 21.7 |
| 11 | 10.7 | 30.0 | 11.4 | 29.0 | 13.4 | 26.9 |
| 22 | 11.3 | 39.5 | 15.7 | 40.2 | 13.6 | 33.9 |
| 23 | 13.2 | 35.5 | 20.5 | 37.2 | 18.1 | 35.2 |
| 24 | 9.2 | 24.4 | 14.3 | 26.0 | 11.8 | 18.1 |
| 25 | 12.8 | 33.5 | 16.1 | 29.4 | 11.9 | 25.1 |
| 26 | 11.5 | 31.2 | 15.8 | 30.8 | 17.1 | 29.4 |
| 28 | 9.9 | 24.7 | 11.0 | 19.0 | 11.6 | 23.8 |
| 29 | 12.2 | 37.1 | 15.8 | 34.8 | — | — |
| 37 | 14.4 | 37.9 | 15.6 | 38.1 | 15.5 | 36.4 |
| 38 | 16.1 | 40.0 | 14.3 | 29.7 | 10.9 | 24.7 |
| 39 | 11.8 | 29.3 | 13.1 | 28.4 | 11.5 | 24.1 |
| 40 | 11.4 | 24.0 | 16.5 | 35.4 | 16.0 | 28.5 |
| 41 | 13.5 | 32.8 | 22.3 | 35.2 | 17.9 | 25.4 |
| 42 | 11.1 | 30.0 | 14.2 | 27.4 | — | — |
| 43 | 12.0 | 34.8 | 12.9 | 26.3 | 11.4 | 23.5 |
| 44 | 15.1 | 33.1 | 18.1 | 35.0 | 20.2 | 31.5 |
| 45 | 12.4 | 30.4 | 16.5 | 24.2 | 12.9 | 24.6 |
| 46 | 11.5 | 31.6 | 17.3 | 27.6 | 19.9 | 31.3 |

TABLE 12.1
Transepidermal water loss values (E, g m⁻² h⁻¹) for the control

| | | | |
|---|---|---|---|
| Volunteer | D0 | D14 | D28 |

| Code | T0 | T30 | T0 | T30 | T0 | T30 |
|---|---|---|---|---|---|---|
| 1 | 13.2 | 30.1 | 12.0 | 31.7 | 13.6 | 32.2 |
| 2 | 10.3 | 22.6 | 10.8 | 30.2 | 13.5 | 30.0 |
| 3 | 14.0 | 33.1 | 11.8 | 30.0 | 13.2 | 32.2 |
| 4 | 10.5 | 36.6 | 11.0 | 31.0 | 11.2 | 35.1 |
| 5 | 13.6 | 28.8 | 17.0 | 35.0 | 20.7 | 36.4 |
| 6 | 12.9 | 33.2 | 17.8 | 34.8 | 15.0 | 31.1 |
| 7 | 11.2 | 27.5 | 7.6 | 24.9 | 11.2 | 29.2 |
| 8 | 12.8 | 34.1 | 10.9 | 31.9 | 12.8 | 30.2 |
| 9 | 14.1 | 36.1 | 19.2 | 35.0 | 14.1 | 31.9 |
| 10 | 6.4 | 18.5 | 8.2 | 19.2 | 7.6 | 23.4 |
| 11 | 11.0 | 31.2 | 13.6 | 37.2 | 11.6 | 27.5 |
| 22 | 10.5 | 35.9 | 15.0 | 41.2 | 12.5 | 34.7 |
| 23 | 16.1 | 35.5 | 17.2 | 38.8 | 17.7 | 38.8 |
| 24 | 11.0 | 27.2 | 15.4 | 29.3 | 9.4 | 20.7 |
| 25 | 13.4 | 31.8 | 17.1 | 33.8 | 15.4 | 32.6 |
| 26 | 10.8 | 33.5 | 19.0 | 35.1 | 15.5 | 35.2 |
| 28 | 10.0 | 22.2 | 8.9 | 18.9 | 15.2 | 25.7 |
| 29 | 11.5 | 34.0 | 17.5 | 30.9 | — | — |
| 37 | 13.2 | 39.2 | 15.5 | 39.8 | 14.4 | 44.9 |
| 38 | 12.7 | 33.9 | 14.0 | 30.5 | 12.7 | 30.2 |
| 39 | 11.5 | 26.2 | 12.1 | 29.7 | 12.6 | 27.9 |
| 40 | 10.5 | 24.7 | 13.1 | 39.7 | 15.5 | 35.7 |
| 41 | 12.0 | 29.6 | 22.2 | 40.3 | 14.7 | 27.4 |
| 42 | 11.9 | 28.3 | 13.4 | 31.5 | — | — |
| 43 | 13.4 | 39.0 | 16.7 | 37.6 | 12.5 | 29.6 |
| 44 | 11.4 | 27.0 | 15.9 | 32.1 | 15.2 | 29.0 |
| 45 | 12.8 | 28.0 | 13.5 | 25.9 | 10.9 | 26.7 |
| 46 | 11.3 | 28.6 | 16.1 | 30.1 | 17.3 | 32.5 |

TABLE 12.2
ΔE Values (Equation 1)

| | D0 | | D14 | | D28 | |
|---|---|---|---|---|---|---|
| Volunteer Code | Control | Product | Control | Product | Control | Control |
| 1 | 19.8 | 16.9 | 15.3 | 19.7 | 14.1 | 18.6 |
| 2 | 15.0 | 12.3 | 18.8 | 19.4 | 16.1 | 16.5 |
| 3 | 21.9 | 19.1 | 26.3 | 18.2 | 16.7 | 19.0 |
| 4 | 23.7 | 26.1 | 20.2 | 20.0 | 20.3 | 23.9 |
| 5 | 13.1 | 15.2 | 15.3 | 18.0 | 15.0 | 15.7 |
| 6 | 17.6 | 20.3 | 15.1 | 17.0 | 15.6 | 16.1 |
| 7 | 19.3 | 16.3 | 19.1 | 17.3 | 19.1 | 18.0 |
| 8 | 18.6 | 21.3 | 18.6 | 21.0 | 17.6 | 17.4 |
| 9 | 24.9 | 22.0 | 22.4 | 15.8 | 16.2 | 17.8 |
| 10 | 10.8 | 12.1 | 10.3 | 11.0 | 9.0 | 15.8 |
| 11 | 19.3 | 20.2 | 17.6 | 23.6 | 13.5 | 15.9 |
| 22 | 28.2 | 25.4 | 24.5 | 26.2 | 20.3 | 22.2 |
| 23 | 22.3 | 19.4 | 16.7 | 21.6 | 17.1 | 21.1 |
| 24 | 15.2 | 16.2 | 11.7 | 13.9 | 6.3 | 11.3 |
| 25 | 20.7 | 18.4 | 13.3 | 16.7 | 13.2 | 17.2 |
| 26 | 19.7 | 22.7 | 15.0 | 16.1 | 12.3 | 19.7 |
| 28 | 14.8 | 12.2 | 8.0 | 10.0 | 12.2 | 10.5 |
| 29 | 24.9 | 22.5 | 19.0 | 13.4 | — | — |
| 37 | 23.5 | 26.0 | 22.5 | 24.3 | 20.9 | 30.5 |
| 38 | 23.9 | 21.2 | 15.4 | 16.5 | 13.8 | 17.5 |
| 39 | 17.5 | 14.7 | 15.3 | 17.6 | 12.6 | 15.3 |
| 40 | 12.6 | 14.2 | 18.9 | 26.6 | 12.5 | 20.2 |
| 41 | 19.3 | 17.6 | 12.9 | 18.1 | 7.5 | 12.7 |
| 42 | 18.9 | 16.4 | 13.2 | 18.1 | — | — |
| 43 | 22.8 | 25.6 | 13.4 | 20.9 | 12.1 | 17.1 |
| 44 | 18.0 | 15.6 | 16.9 | 16.2 | 11.3 | 13.8 |
| 45 | 18.0 | 15.2 | 7.7 | 12.4 | 11.7 | 15.8 |
| 46 | 20.1 | 17.3 | 10.3 | 14.0 | 11.4 | 15.2 |

TABLE 12.3
RE Values (Equation 2)

| Volunteer | Product | | Control | |
|---|---|---|---|---|
| Code | D14 | D28 | D14 | D28 |
| 1 | 0.77 | 0.71 | 1.17 | 1.10 |
| 2 | 1.25 | 1.07 | 1.58 | 1.34 |
| 3 | 1.20 | 0.76 | 0.95 | 0.99 |
| 4 | 0.85 | 0.86 | 0.77 | 0.92 |
| 5 | 1.17 | 1.15 | 1.18 | 1.03 |
| 6 | 0.86 | 0.89 | 0.84 | 0.79 |
| 7 | 0.99 | 0.99 | 1.06 | 1.10 |
| 8 | 1.00 | 0.95 | 0.99 | 0.82 |
| 9 | 0.90 | 0.65 | 0.72 | 0.81 |
| 10 | 0.95 | 0.83 | 0.91 | 1.31 |
| 11 | 0.91 | 0.70 | 1.17 | 0.79 |
| 22 | 0.87 | 0.72 | 1.03 | 0.87 |
| 23 | 0.75 | 0.77 | 1.11 | 1.09 |
| 24 | 0.77 | 0.41 | 0.86 | 0.70 |
| 25 | 0.64 | 0.64 | 0.91 | 0.93 |
| 26 | 0.76 | 0.62 | 0.71 | 0.87 |
| 28 | 0.54 | 0.82 | 0.82 | 0.86 |
| 29 | 0.76 | — | 0.60 | — |
| 37 | 0.96 | 0.89 | 0.93 | 1.17 |
| 38 | 0.64 | 0.58 | 0.78 | 0.83 |
| 39 | 0.87 | 0.72 | 1.20 | 1.04 |
| 40 | 1.50 | 0.99 | 1.87 | 1.42 |
| 41 | 0.67 | 0.39 | 1.03 | 0.72 |
| 42 | 0.70 | — | 1.10 | — |
| 43 | 0.59 | 0.53 | 0.82 | 0.67 |
| 44 | 0.94 | 0.63 | 1.04 | 0.88 |
| 45 | 0.43 | 0.65 | 0.82 | 1.04 |
| 46 | 0.51 | 0.57 | 0.81 | 0.88 |

TABLE 12.4
FB Values (Equation 3)

| Volunteer Code | D14 | D28 |
|---|---|---|
| 01 | 39.30 | 38.85 |
| 02 | 32.39 | 26.81 |
| 03 | −24.80 | 23.22 |
| 04 | −8.60 | 5.92 |
| 05 | 1.63 | −11.21 |
| 06 | −2.05 | −9.33 |
| 07 | 7.17 | 11.47 |
| 08 | −1.41 | −12.93 |
| 09 | −18.14 | 15.85 |
| 10 | −4.46 | 47.25 |
| 11 | 25.64 | 8.76 |
| 22 | 16.27 | 15.42 |
| 23 | 36.45 | 32.08 |
| 24 | 8.83 | 28.31 |
| 25 | 26.51 | 29.71 |
| 26 | −5.22 | 24.35 |
| 28 | 27.91 | 3.63 |
| 29 | −16.75 | — |
| 37 | −2.28 | 28.37 |
| 38 | 13.40 | 24.81 |
| 39 | 32.30 | 32.08 |
| 40 | 37.32 | 43.05 |
| 41 | 36.00 | 33.30 |
| 42 | 40.52 | — |
| 43 | 22.87 | 13.73 |
| 44 | 9.96 | 25.68 |
| 45 | 38.80 | 38.95 |
| 46 | 29.68 | 31.14 |

FIG. 7 shows the variation average of the transepidermal water loss (ΔEDi) in relation to time (i=0, 14 or 28 days) for forearms onto which the product was applies and for the respective control forearm.

5.4.1. Basal Homogeneity

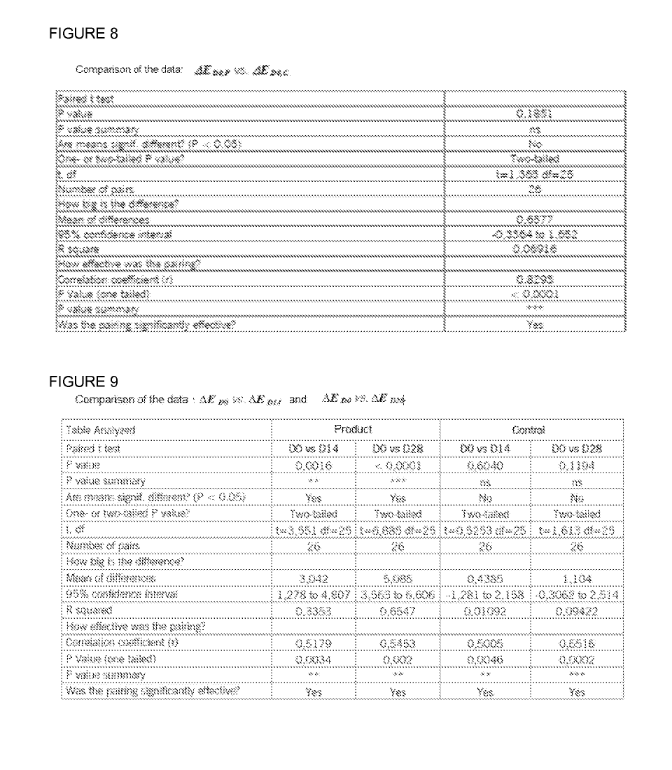

Table 13 summarizes the results obtained from the statistical analysis of basal data homogeneity (FIG. 8).

TABLE 13
summary of the results obtained from the statistical analysis of
basal data homogeneity

| Comparison Group: | Parameter: D0 |
|---|---|
| ΔE _{Product} vs. ΔE _{Control} | 0.1851 (non-significant) |

According to the obtained results there was no statistically significant difference (P>0.05) among the basal values of variation in transepidermal water loss between the forearms onto which the product was applied and the respective control forearms.

5.4.2. Significance of the Effect

Table 14 displays the results obtained from the statistical evaluation of the significance of variations in the transepidermal water loss values throughout the study (FIG. 9).

TABLE 14
Summarized data on the statistical analysis of the significance of
the changes in the skin barrier. P values

| Comparison Group: | Control | Mixture 1 |
|---|---|---|
| ΔE D0 vs. ΔE D14 | 0.6040 (non-significant) | 0.0016 (significant) |
| ΔE D0 vs. ΔE D28 | 0.1194 (non-significant) | <0.0001 (significant) |

According to the results obtained for the control, there were observed no significant variations in the TEWL values after 14 and 28 days, indicating that there were no significant changes in the skin barrier throughout the study.

For MIXTURE 1, a significant reduction in the TEWL variation values after 14 and 28 days of continuous use was noticed, which indicates that the use of said product resulted in significant changes in the skin barrier with respect to the strengthening thereof.

5.4.3. Comparison between product and control

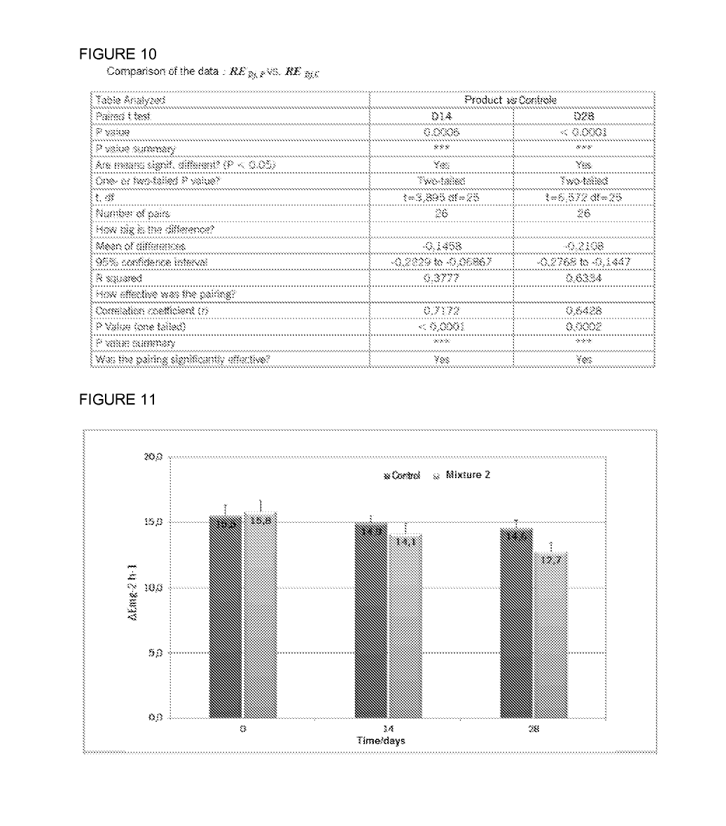

The results of the statistical analysis for assessment of the significance of the effect of the product in relation to the control are displayed in Table 15 and FIG. 10

TABLE 15
Statistical analysis: product vs. control. P values

| Comparison Group | D14 | D28 |
|---|---|---|
| RE_{P} vs. RE_{C} | 0.0006 (significant) | <0.0001 (significant) |

According to the obtained results, after 14 and 28 days of continuous use of MIXTURE 1, the observed reduction of the TEWL values was significantly superior to that observed for the control, indicating that the use of the product provided a significant strengthening effect on the skin barrier already after 14 days of use.

MIXTURE 1 provided a significant strengthening effect on the skin barrier (FB), in relation to the initial condition of the skin and control, of 14% after 14 days of use and 21% after 28 days of use.

5.5. Analysis of Daily Use

77% of the volunteers used the product correctly.

19% of the volunteers stopped using the product for 1 or 2 days, stating they had forgotten about it.

4% of the volunteers stopped using the product for 3 days, stating they had forgotten about it.

### 6. Conclusion

According to the study conditions disclosed, it is concluded that MIXTURE 1 applied to the skin in the region of the forearm significantly strengthened the skin barrier when compared to the control (skin free of any products) after 14 and 28 days of continuous use.

The skin barrier strengthening percentage values compared to the initial state of the skin and to the control were of 14% after 14 days of use and 21% after 28 days of use.

### TEST 3—Evaluation of the Strengthening of Skin Barrier Caused by the Use of Cosmetic Product

### 1. Objective

To evaluate the strengthening effect of the skin barrier caused by the home use of MIXTURE 2 during 28 days by using a process for removing layers of the stratum corneum with 30 consecutive applications and removals of adhesive tape and evaluate the transepidermal water loss by evaporimetry.

### 2. Table of Volunteers

The volunteers were instructed to discontinue the use of any topical product in the region of the forearms 48 hours prior to the beginning of the study. They also received instructions regarding the hours the trials were to be conducted and were informed not to use any products during the period of the study other than the provided product.

### 3. Procedures for Conducting Evaluations

3.1. Climatization

Prior to the beginning of the evaluation, the volunteers remained in the laboratory with the forearms exposed and at rest at 20±2° C. and 50±5% relative humidity for at least 30 minutes.

3.2. Measuring area

In each volunteer's anterior surface of the forearm (right and left) an area of 2.5×4.0 cm was marked with the aid of a guide and a surgical pen, and the arm for application of the product and the control arm were ordered alternately, as one can see in Table 16 below.

TABLE 16

| | | | Area of application | |
|---|---|---|---|---|
| Volunteers | Age | Phototype | Right forearm | Left forearm |
| 12 | 58 | II | Mixture 2 | Control |
| 13 | 43 | III | Control | Mixture 2 |
| 15 | 41 | IV | Control | Mixture 2 |
| 16 | 50 | III | Mixture 2 | Control |
| 17 | 32 | III | Control | Mixture 2 |
| 18 | 28 | III | Mixture 2 | Control |
| 19 | 40 | IV | Control | Mixture 2 |
| 20 | 40 | IV | Mixture 2 | Control |
| 21 | 28 | IV | Control | Mixture 2 |
| 30 | 59 | IV | Mixture 2 | Control |
| 31 | 36 | IV | Control | Mixture 2 |
| 32 | 43 | III | Mixture 2 | Control |
| 33 | 43 | III | Control | Mixture 2 |
| 34 | 49 | III | Mixture 2 | Control |
| 35 | 55 | II | Control | Mixture 2 |
| 36 | 38 | IV | Mixture 2 | Control |
| 47 | 42 | III | Control | Mixture 2 |
| 48 | 55 | III | Mixture 2 | Control |
| 49 | 42 | III | Control | Mixture 2 |
| 50 | 47 | IV | Mixture 2 | Control |
| 51 | 49 | IV | Control | Mixture 2 |
| 52 | 32 | III | Mixture 2 | Control |
| 53 | 29 | V | Control | Mixture 2 |
| 54 | 49 | V | Mixture 2 | Control |
| 55 | 42 | III | Control | Mixture 2 |
| 56 | 51 | IV | Mixture 2 | Control |
| 57 | 39 | III | Control | Mixture 2 |
| 58 | 40 | III | Mixture 2 | Control |

3.3. Equipment

The same aspects of TEST 2 apply to TEST 3.

3.4. Taking Measurements

The same aspects of TEST 2 apply to TEST 3.

3.5. Applying the Product

The same aspects of TEST 2 apply to TEST 3.

### 4. Data Analysis and Interpretation

4.1. Software for Obtaining Average Values and Data Analysis:

The same aspects of TEST 2 apply to TEST 3.

4.2. Software for Statistical Analysis:

The same aspects of TEST 2 apply to TEST 3.

4.3. Interpreting the Results

The same aspects of TEST 2 apply to TEST 3.

4.3.1. Calculations

The same aspects of TEST 2 apply to TEST 3.

4.3.2. Statistical Evaluations

4.3.2.1. Basal Homogeneity

The same aspects of TEST 2 apply to TEST 3.

4.3.2.2. Significance of the Effect

The same aspects of TEST 2 apply to TEST 3.

4.3.2.3. Comparison Between Product and Control

The same aspects of TEST 2 apply to TEST 3.

### 5. Results and Discussions.

5.1. Statistics on the Participations of Volunteers

Total contacted volunteers: 81;

Total of acceptances: 46 (57% of the contacted volunteers);

Total absences in the day of the study: 17;

Total volunteers dismissed after evaluation of inclusion and exclusion criteria: 1;

Effectively included volunteers: 28 (61% of the acceptances);

Volunteers who completed the study: 26 (93%).

5.2. General Data on the Study Group:

Average age: 43±9 years.

5.3. Climate Control

Statistical data on the environmental monitoring throughout the days the study was carried out at the laboratory for evaluation and climatization of the volunteers:

Day 1 (08:00 a.m. to 06:00 p.m.):

Temperature: (20.6±0.4° C. (95% Confidence interval: 20.5° C. to 20.8° C.)

Relative air humidity: (53±1) % (95% Confidence interval: 53% to 54%)

Day 2 (08:00 a.m. to 06:00 p.m.):

Temperature: (20.8±0.3° C. (95% Confidence interval: 20.6° C. to 21.0° C.)

Relative air humidity: (52±2) % (95% Confidence interval: 51% to 53%)

Day 3 (08:00 a.m. to 06:00 p.m.):

Temperature: (21.2±0.4° C. (95% Confidence interval: 21.0° C. to 21.3° C.)

Relative air humidity: (50±2) % (95% Confidence interval: 49% to 51%)

Day 4 (08:00 a.m. to 06:00 p.m.):

Temperature: (20.5±0.4° C. (95% Confidence interval: 20.4° C. to 20.7° C.)

Relative air humidity: (54±1) % (95% Confidence interval: 54% to 55%)

Day 5 (08:00 a.m. to 06:00 p.m.):

Temperature: (19.8±1.1° C. (95% Confidence interval: 19.3° C. to 20.3° C.)

Relative air humidity: (55±4) % (95% Confidence interval: 54% to 57%)

Day 6 (08:00 a.m. to 06:00 p.m.):

Temperature: (20.6±0.6° C. (95% Confidence interval: 20.4° C. to 20.9° C.)

Relative air humidity: (55±4) % (95% Confidence interval: 54% to 57%)

Day 7 (08:00 a.m. to 06:00 p.m.):

Temperature: (21.0±0.4° C. (95% Confidence interval: 20.8° C. to 21.1° C.)

Relative air humidity: (49±3) % (95% Confidence interval: 48% to 50%)

Day 8 (08:00 a.m. to 06:00 p.m.):

Temperature: (21.2±0.6° C. (95% Confidence interval: 20.9° C. to 21.4° C.)

Relative air humidity: (53±3) % (95% Confidence interval: 52% to 54%) Day 9 (08:00 a.m. to 06:00 p.m.):

Temperature: (21.4±0.7° C. (95% Confidence interval: 21.1° C. to 21.7° C.)

Relative air humidity: (48±3) % (95% Confidence interval: 46% to 49%)

According to the data registered in the climate control, the temperature and humidity in the laboratory for measurements and climatization of the volunteers remained within the range established in the study protocol on all evaluation days.

5.4. Results Obtained from the Evaluation:

The raw data obtained from transepidermal water loss are listed in Tables 17 to 17.4, as well as the calculated parameters: ΔE (Equation 1), RE (Equation 2) and FB (Equation 3).

TABLE 17
Transepidermal water loss values (E, g m⁻² h⁻¹) for MIXTURE 2

| | | | |
|---|---|---|---|
| Volunteer | D0 | D14 | D28 |

| code | T30 | T0 | T30 | T0 | T30 | T30 |
|---|---|---|---|---|---|---|
| 12 | 15.7 | 40.7 | 20.9 | 43.3 | 18.2 | 34.0 |
| 13 | 15.8 | 35.6 | 17.5 | 38.5 | 17.7 | 35.9 |
| 15 | 14.5 | 32.9 | 17.2 | 35.4 | 16.4 | 28.1 |
| 16 | 16.0 | 27.5 | 22.0 | 33.3 | 16.7 | 24.1 |
| 17 | 11.9 | 21.3 | 12.8 | 23.4 | 14.3 | 23.7 |
| 18 | 10.4 | 26.7 | 11.7 | 25.3 | — | — |
| 19 | 13.7 | 23.5 | 14.4 | 23.0 | 14.7 | 22.0 |
| 20 | 13.5 | 26.0 | 13.6 | 24.4 | 12.1 | 23.2 |
| 21 | 14.0 | 26.6 | 13.1 | 25.2 | 13.9 | 26.2 |
| 30 | 12.8 | 34.0 | 11.2 | 27.7 | 11.3 | 26.6 |
| 31 | 13.2 | 33.6 | 13.7 | 27.3 | 13.4 | 25.8 |
| 32 | 12.8 | 31.2 | 15.2 | 36.1 | 17.1 | 30.0 |
| 33 | 12.4 | 30.2 | 14.5 | 30.5 | 14.7 | 30.0 |
| 34 | 14.5 | 31.2 | 12.3 | 24.4 | 12.0 | 24.1 |
| 35 | 14.6 | 31.9 | 13.3 | 26.8 | 13.5 | 27.0 |
| 36 | 13.3 | 37.8 | 18.0 | 31.4 | 15.3 | 30.6 |
| 47 | 11.4 | 33.9 | 11.1 | 27.0 | 11.3 | 28.8 |
| 48 | 14.5 | 27.8 | 16.1 | 26.6 | 15.8 | 25.2 |
| 49 | 13.4 | 23.7 | 16.0 | 22.7 | 15.7 | 24.6 |
| 50 | 12.5 | 25.3 | 13.8 | 24.6 | 13.5 | 24.1 |
| 51 | 13.8 | 30.5 | 17.0 | 33.4 | 16.6 | 32.6 |
| 52 | 12.0 | 21.0 | 14.0 | 22.1 | 13.4 | 20.8 |
| 53 | 12.7 | 26.3 | 12.7 | 27.9 | 12.5 | 27.5 |
| 54 | 15.4 | 27.5 | — | — | — | — |
| 55 | 11.8 | 26.0 | 10.1 | 24.4 | 10.3 | 22.6 |
| 56 | 13.7 | 27.5 | 12.2 | 30.4 | 12.5 | 29.9 |
| 57 | 15.9 | 33.2 | 14.2 | 31.1 | 14.3 | 29.4 |
| 58 | 12.7 | 23.7 | 12.8 | 25.8 | 12.6 | 24.3 |

TABLE 17.1
Transepidermal water loss values (E, g m⁻² h⁻¹) for the control

| | | | |
|---|---|---|---|
| Volunteer | D0 | D14 | D28 |

| code | T30 | T0 | T30 | T0 | T30 | T30 |
|---|---|---|---|---|---|---|
| 12 | 16.1 | 42.8 | 22.3 | 44.3 | 21.0 | 42.8 |
| 13 | 13.2 | 30.3 | 13.1 | 30.5 | 18.6 | 33.5 |
| 15 | 13.8 | 30.4 | 21.6 | 37.2 | 16.3 | 32.1 |
| 16 | 16.7 | 30.0 | 18.5 | 29.9 | 14.9 | 23.3 |
| 17 | 10.9 | 21.9 | 10.3 | 24.3 | 10.1 | 24.2 |
| 18 | 12.1 | 27.9 | 12.7 | 26.7 | — | — |
| 19 | 11.2 | 20.7 | 13.2 | 25.5 | 14.8 | 27.3 |
| 20 | 15.1 | 30.4 | 16.6 | 27.9 | 17.0 | 29.0 |
| 21 | 13.9 | 27.0 | 14.3 | 26.9 | 15.6 | 26.7 |
| 30 | 11.8 | 30.9 | 15.4 | 32.5 | 15.4 | 30.2 |
| 31 | 11.1 | 28.5 | 16.9 | 30.2 | 17.1 | 31.0 |
| 32 | 13.4 | 30.0 | 11.9 | 27.1 | 12.1 | 27.1 |
| 33 | 12.9 | 28.4 | 11.1 | 25.0 | 13.5 | 24.1 |
| 34 | 16.2 | 33.4 | 17.2 | 33.6 | 17.2 | 33.0 |
| 35 | 15.6 | 30.4 | 18.2 | 32.3 | 18.0 | 31.9 |
| 36 | 14.7 | 36.3 | 16.2 | 32.8 | 14.8 | 33.0 |
| 47 | 11.6 | 35.2 | 13.8 | 33.3 | 14.2 | 35.8 |
| 48 | 14.4 | 25.3 | 15.9 | 30.3 | 16.7 | 30.5 |
| 49 | 13.6 | 26.3 | 18.9 | 28.9 | 17.8 | 29.7 |
| 50 | 12.7 | 23.9 | 13.0 | 26.1 | 14.1 | 27.1 |
| 51 | 12.5 | 27.7 | 17.4 | 35.0 | 18.2 | 33.2 |
| 52 | 12.1 | 23.3 | 14.2 | 24.0 | 14.5 | 24.2 |
| 53 | 10.2 | 26.4 | 14.2 | 30.0 | 14.7 | 31.8 |
| 54 | 15.9 | 26.2 | — | — | — | — |
| 55 | 10.5 | 24.7 | 13.3 | 27.2 | 14.8 | 29.2 |
| 56 | 14.0 | 25.2 | 14.8 | 32.2 | 16.2 | 32.9 |
| 57 | 12.5 | 31.8 | 15.4 | 32.3 | 15.7 | 32.9 |
| 58 | 13.9 | 25.9 | 15.1 | 31.4 | 15.5 | 30.9 |

TABLE 17.2
ΔE values (Equation 1)

| | D0 | | D14 | | D28 | |
|---|---|---|---|---|---|---|
| Volunteer code | Product | Control | Product | Control | Product | Control |
| 12 | 25.0 | 26.7 | 22.4 | 22.0 | 15.8 | 21.8 |
| 13 | 19.8 | 17.1 | 21.0 | 17.4 | 18.2 | 14.9 |
| 15 | 18.4 | 16.6 | 18.2 | 15.6 | 11.7 | 15.8 |
| 16 | 11.5 | 13.3 | 11.3 | 11.4 | 7.4 | 8.4 |
| 17 | 9.4 | 11.0 | 10.6 | 14.0 | 9.4 | 14.1 |
| 18 | 16.3 | 15.8 | 13.6 | 14.0 | — | — |
| 19 | 9.8 | 9.5 | 8.6 | 12.3 | 7.3 | 12.5 |
| 20 | 12.5 | 15.3 | 10.8 | 11.3 | 11.1 | 12.0 |
| 21 | 12.6 | 13.1 | 12.1 | 12.6 | 12.3 | 11.1 |
| 30 | 21.2 | 19.1 | 16.5 | 17.1 | 15.3 | 14.8 |
| 31 | 20.4 | 17.4 | 13.6 | 13.3 | 12.4 | 13.9 |
| 32 | 18.4 | 16.6 | 20.9 | 15.2 | 12.9 | 15.0 |
| 33 | 17.8 | 15.5 | 16.0 | 13.9 | 15.3 | 10.6 |
| 34 | 16.7 | 17.2 | 12.1 | 16.4 | 12.1 | 15.8 |
| 35 | 17.3 | 14.8 | 13.5 | 14.1 | 13.5 | 13.9 |
| 36 | 24.5 | 21.6 | 13.4 | 16.6 | 15.3 | 18.2 |
| 47 | 22.5 | 23.6 | 15.9 | 19.5 | 17.5 | 21.6 |
| 48 | 13.3 | 10.9 | 10.5 | 14.4 | 9.4 | 13.8 |
| 49 | 10.3 | 12.7 | 6.7 | 10.0 | 8.9 | 11.9 |
| 50 | 12.8 | 11.2 | 10.8 | 13.1 | 10.6 | 13.0 |
| 51 | 16.7 | 15.2 | 16.4 | 17.6 | 16.0 | 15.0 |
| 52 | 9.0 | 11.2 | 8.1 | 9.8 | 7.4 | 9.7 |
| 53 | 13.6 | 16.2 | 15.2 | 15.8 | 15.0 | 17.1 |
| 54 | 12.1 | 10.3 | — | — | — | — |
| 55 | 14.2 | 14.2 | 14.3 | 13.9 | 12.3 | 14.4 |
| 56 | 13.8 | 11.2 | 18.2 | 17.4 | 17.4 | 16.7 |
| 57 | 17.3 | 19.3 | 16.9 | 16.9 | 15.1 | 17.2 |
| 58 | 11.0 | 12.0 | 13.0 | 16.3 | 11.7 | 15.4 |

TABLE 17.3
RE values (Equation 2)

| | | |
|---|---|---|
| Volunteer | Product | Control |

| code | D14 | D28 | D14 | D28 |
|---|---|---|---|---|
| 12 | 0.90 | 0.63 | 0.82 | 0.82 |
| 13 | 1.06 | 0.92 | 1.02 | 0.87 |
| 15 | 0.99 | 0.64 | 0.94 | 0.95 |
| 16 | 0.98 | 0.64 | 0.86 | 0.63 |
| 17 | 1.13 | 1.00 | 1.27 | 1.28 |
| 18 | 0.83 | — | 0.89 | — |
| 19 | 0.88 | 0.74 | 1.29 | 1.32 |
| 20 | 0.86 | 0.89 | 0.74 | 0.78 |
| 21 | 0.96 | 0.98 | 0.96 | 0.85 |
| 30 | 0.78 | 0.72 | 0.90 | 0.77 |
| 31 | 0.67 | 0.61 | 0.76 | 0.80 |
| 32 | 1.14 | 0.70 | 0.92 | 0.90 |
| 33 | 0.90 | 0.86 | 0.90 | 0.68 |
| −34 | 0.72 | 0.72 | 0.95 | 0.92 |
| 35 | 0.78 | 0.78 | 0.95 | 0.94 |
| 36 | 0.55 | 0.62 | 0.77 | 0.84 |
| 47 | 0.71 | 0.78 | 0.83 | 0.92 |
| 48 | 0.79 | 0.71 | 1.32 | 1.27 |
| 49 | 0.65 | 0.86 | 0.79 | 0.94 |
| 50 | 0.84 | 0.83 | 1.17 | 1.16 |
| 51 | 0.98 | 0.96 | 1.16 | 0.99 |
| 52 | 0.90 | 0.82 | 0.88 | 0.87 |
| 53 | 1.12 | 1.10 | 0.98 | 1.06 |
| 55 | 1.01 | 0.87 | 0.98 | 1.01 |
| 56 | 1.32 | 1.26 | 1.55 | 1.49 |
| 57 | 0.98 | 0.87 | 0.88 | 0.89 |
| 58 | 1.18 | 1.06 | 1.36 | 1.28 |

TABLE 17.4
FB values (Equation 3)

| Volunteer code | D14 | D28 |
|---|---|---|
| 12 | −7.20 | 18.45 |
| 13 | −4.31 | −4.78 |
| 15 | −4.94 | 31.59 |
| 16 | −12.55 | −1.19 |
| 17 | 14.51 | 28.18 |
| 18 | 5.17 | — |
| 19 | 41.72 | 57.09 |
| 20 | −12.54 | −10.37 |
| 21 | 0.15 | −12.89 |
| 30 | 11.70 | 5.32 |
| 31 | 9.77 | 19.10 |
| 32 | −22.02 | 20.25 |
| 33 | −0.21 | −17.57 |
| 34 | 22.89 | 19.41 |
| 35 | 17.24 | 15.88 |
| 36 | 22.16 | 21.81 |
| 47 | 11.96 | 13.75 |
| 48 | 53.16 | 55.93 |
| 49 | 13.69 | 7.29 |
| 50 | 32.59 | 33.26 |
| 51 | 17.59 | 2.88 |
| 52 | −2.50 | 4.38 |
| 53 | −14.23 | −4.74 |
| 55 | −2.82 | 14.79 |
| 56 | 23.47 | 23.02 |
| 57 | −10.12 | 1.84 |
| 58 | 17.65 | 21.97 |
| 12 | −7.20 | 18.45 |

FIG. 10 illustrates the averages of variation in transepidermal water loss (ΔE_{Di}) according to time (i=0.14 or 28 days) for the forearms wherein the product was applied and for the respective control forearms.

5.4.1. Basal Homogeneity

Table 18 summarizes the Results obtained from the statistical analysis of basal homogeneity of the data, listed in FIG. 11.

TABLE 18
Summarized data of the statistical analysis. P values.

| Comparison group | Parameter: D0 |
|---|---|
| ΔE_{Product} vs. ΔE_{Control} | 0.4873 (non-significant) |

According to the obtained results there was no statistically significant difference (P>0.05) among the basal values of variation in transepidermal water loss between the forearms wherein the product was applied and the respective control forearms.

5.4.2. Significance of the Effect

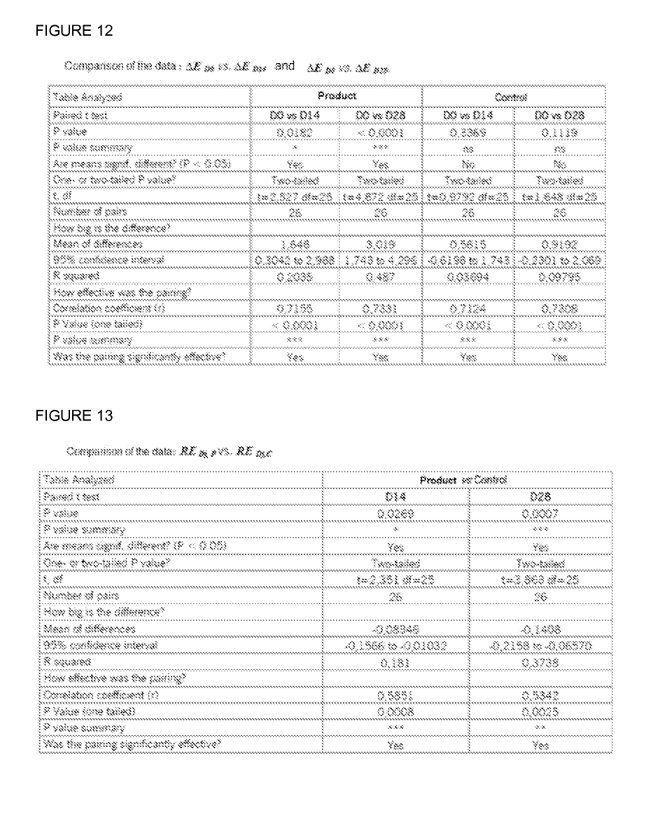

Table 19 discloses the results obtained from the statistical evaluation of the variations in the transepidermal water loss values throughout the study (FIG. 12).

TABLE 19
discloses the Results obtained from the statistical evaluation of the
variations in the transepidermal water loss values throughout the study.

| Comparison group | Control | Mixture 2 |
|---|---|---|
| ΔE D0 vs. ΔE D14 | 0.3369 (non-significant) | 0.0182 (significant) |
| ΔE D0 vs. ΔE D28 | 0.1119 (non-significant) | <0.0001 (significant) |

According to the results obtained for the control, there were observed no significant variations in the TEWL values after 14 and 28 days, indicating that there were no significant changes in the skin barrier throughout the study.

For MIXTURE 2 it was noted a significant reduction in the TEWL variation values after 14 and 28 days of continuous use, indicating that the use of this product provided significant changes in the skin barrier towards its strengthening.

5.4.3. Comparison Between Product and Control

The results of the statistical analysis for assessment of the significance of the effect of the product in relation to the control are displayed in Table 20 and FIG. 13.

TABLE 20
Statistical analysis: product vs. control. P values.

| Comparison group | D14 | D28 |
|---|---|---|
| RE_{P} vs. RE_{C} | 0.0269 (significant) | <0.0007 (significant) |

According to the obtained results, after 14 and 28 days of continuous use of MIXTURE 2, the observed reduction of the TEWL values was significantly superior to that observed for the control, indicating that the use of the product provided a significant strengthening effect on the skin barrier already after 14 days of use.

MIXTURE 2 provided a significant strengthening effect on the skin barrier (FB), in relation to the initial skin condition and to the control, of 9% after 14 days of use and 14% after 28 days of use.

5.5. Analysis of Daily Use

85% of the volunteers used the product correctly.

15% of the volunteers stopped using the product for 1 or 2 days, stating they had forgotten about it.

### 6. Conclusion

According to the study conditions disclosed, it is concluded that MIXTURE 2 applied to the skin in the region of the forearm significantly strengthened the skin barrier when compared to the control (skin free of any products) after 14 and 28 days of continuous use.

The skin barrier strengthening percentage values compared to the initial state of the skin and to the control were of 9% after 14 days of use and 14% after 28 days of use.

### TEST 4—Evaluation of the Skin Barrier Strengthening Caused by the Use of Cosmetic Product

### 1. Objective

Evaluate the strengthening effect on the skin barrier caused by the home use of MIXTURE 3 after 14 and 28 days by using a process for removing layers of the stratum corneum with 30 successive applications and removals of the adhesive tape (adhesive tape stripping) and evaluate the transepidermal water loss by evaporimetry.

### 2. Table of Volunteers

The volunteers were instructed to discontinue the use of any topical product in the region of the forearms 48 hours prior to the beginning of the study. They also received instructions regarding the hours the trials were to be conducted and were informed not to use any products during the period of the study other than the provided product.

### 3. Procedures for Conducting Evaluations

3.1. Climatization

Prior to the beginning of the evaluation, the volunteers remained in the laboratory with the forearms exposed and at rest at 20±2° C. and 50±5% relative humidity for at least 30 minutes.

3.2. Measuring Area

In each volunteer's anterior surface of the forearm (right and left) an area of 2.5×4.0 cm was marked with the aid of a guide and a surgical pen, and the arm for application of the product and the control arm were ordered alternately, as one can see in Table 21 below.

TABLE 21

| | | | Area of application | |
|---|---|---|---|---|
| Volunteers | Age | Phototype | Right forearm | Left forearm |
| 59 | 51 | III | Mixture 3 | Control |
| 60 | 52 | II | Control | Mixture 3 |
| 61 | 22 | IV | Mixture 3 | Control |
| 62 | 47 | III | Control | Mixture 3 |
| 63 | 40 | III | Mixture 3 | Control |
| 64 | 42 | IV | Control | Mixture 3 |
| 65 | 38 | IV | Mixture 3 | Control |
| 66 | 38 | IV | Control | Mixture 3 |
| 67 | 53 | III | Mixture 3 | Control |
| 68 | 56 | III | Control | Mixture 3 |
| 69 | 48 | II | Mixture 3 | Control |
| 70 | 56 | IV | Control | Mixture 3 |
| 71 | 58 | IV | Mixture 3 | Control |
| 72 | 59 | II | Mixture 3 | Control |
| 73 | 35 | III | Control | Mixture 3 |
| 74 | 44 | IV | Control | Mixture 3 |
| 76 | 43 | IV | Control | Mixture 3 |
| 78 | 55 | III | Control | Mixture 3 |
| 79 | 42 | III | Mixture 3 | Control |
| 80 | 45 | III | Control | Mixture 3 |
| 81 | 46 | IV | Mixture 3 | Control |
| 82 | 57 | IV | Control | Mixture 3 |
| 83 | 60 | IV | Mixture 3 | Control |
| 84 | 47 | IV | Control | Mixture 3 |
| 85 | 34 | III | Mixture 3 | Control |
| 86 | 53 | III | Control | Mixture 3 |
| 87 | 59 | IV | Mixture 3 | Control |
| 88 | 45 | III | Control | Mixture 3 |
| 89 | 54 | IV | Mixture 3 | Control |
| 90 | 35 | IV | Control | Mixture 3 |

3.3. Equipment

The same aspects of TEST 2 and TEST 3 apply to TEST 4.

3.4. Taking Measurements

The same aspects of TEST 2 and TEST 3 apply to TEST 4.

3.5. Applying the Product

The same aspects of TEST 2 and TEST 3 apply to TEST 4.

### 4. Analysis and Interpretation of the Data

4.1. Software for Obtaining Average Values and Data Analysis:

The same aspects of TEST 2 and TEST 3 apply to TEST 4.

### 4.2. Software for Statistical Analysis:

The same aspects of TEST 2 and TEST 3 apply to TEST 4.

4.3. Interpreting the Results

The same aspects of TEST 2 and TEST 3 apply to TEST 4.

4.3.1. Calculations

The same aspects of TEST 2 and TEST 3 apply to TEST 4.

4.3.2. Statistical Evaluations

4.3.2.1. Basal Homogeneity

The same aspects of TEST 2 and TEST 3 apply to TEST 4.

4.3.2.2. Significance of the Effect

The same aspects of TEST 2 and TEST 3 apply to TEST 4.

4.3.2.3. Comparison Between Product and Control

The same aspects of TEST 2 and TEST 3 apply to TEST 4.

### 5. Results and Discussions.

5.1. Statistics on the Participation of Volunteers

Total contacted volunteers: 72;

Total of acceptances: 47 (65% of the contacted volunteers);

Total absences in the day of the study: 15;

Total volunteers dismissed in the evaluation of inclusion and exclusion criteria: 2;

Effectively included volunteers: 30 (64% of the acceptances);

Volunteers who completed the study: 28 (93%).

5.2. General Data on the Study Group:

Average age: 47±9 years.

5.3. Climate Control

Statistical data on the environmental monitoring throughout the days the study was carried out at the laboratory for evaluation and climatization of the volunteers:

Day 1 (08:00 a.m. to 06:00 p.m.):

Temperature: (21.2±0.4° C. (95% Confidence interval: 21.0° C. to 21.3° C.)

Relative air humidity: (50±2) % (95% Confidence interval: 49% to (51%)

Day 2 (08:00 a.m. to 06:00 p.m.):

Temperature: (21.3±0.4° C. (95% Confidence interval: 21.1° C. to 21.4° C.)

Relative air humidity: (45±2) % (95% Confidence interval: 45% to (45%)

Day 3 (08:00 a.m. to 06:00 p.m.):

Temperature: (19.9±0.5° C. (95% Confidence interval: 19.5° C. to 20.3° C.)

Relative air humidity: (54±2) % (95% Confidence interval: 52% to (55%)

Day 4 (08:00 a.m. to 06:00 p.m.):

Temperature: (20.6±0.6° C. (95% Confidence interval: 20.4° C. to 20.9° C.)

Relative air humidity: (55±3) % (95% Confidence interval: 54% to (57%)

Day 5 (08:00 a.m. to 06:00 p.m.):

Temperature: (21.3±0.8° C. (95% Confidence interval: 20.9° C. to 21.6° C.)

Relative air humidity: (44±2) % (95% Confidence interval: 43% to (45%)

Day 6 (08:00 a.m. to 06:00 p.m.):

Temperature: (21.3±0.2° C. (95% Confidence interval: 21.1° C. to 21.5° C.)

Relative air humidity: (46±1) % (95% Confidence interval: 45% to (47%)

Day 7 (08:00 a.m. to 06:00 p.m.):

Temperature: (22.0±0.6° C. (95% Confidence interval: 21.7° C. to 22.2° C.)

Relative air humidity: (45±3) % (95% Confidence interval: 44% to (47%)

Day 8 (08:00 a.m. to 06:00 p.m.):

Temperature: (20.9±0.9° C. (95% Confidence interval: 20.2° C. to 21.6° C.)

Relative air humidity: (47±2) % (95% Confidence interval: 45% to (48%)

According to the data registered in the climate control, the temperature in the laboratory for measurements and climatization of volunteers remained within the range established in the study protocol on all evaluation days. The humidity was slightly lower, in average 1% lower, considering the minimum of the range established on day 5. This occurred due to external weather condition, where the relative air humidity reached 29% according to the Center for Weather and Climate Research Applied to Agriculture (CEPAGRI—Unicamp).

5.4. Results Obtained from the Evaluation

The raw data obtained from transepidermal water loss are listed in Tables 22 to 22.4, as well as the calculated parameters: ΔE (Equation 1), RE (Equation 2) and FB (Equation 3).

TABLE 22
Transepidermal water loss values (E, g m⁻² h⁻¹) for MIXTURE 3

| | | | |
|---|---|---|---|
| Volunteer | D0 | D14 | D28 |

| Code | T30 | T0 | T30 | T0 | T30 | T30 |
|---|---|---|---|---|---|---|
| 59 | 14.4 | 40.1 | 15.1 | 33.2 | 15.0 | 33.9 |
| 60 | 11.3 | 25.3 | 16.5 | 26.3 | 15.3 | 25.3 |
| 61 | 11.3 | 24.3 | 10.5 | 20.0 | 10.0 | 20.2 |
| 62 | 10.7 | 19.6 | 12.6 | 20.8 | 11.7 | 20.4 |
| 64 | 16.9 | 32.1 | 18.6 | 31.8 | 17.7 | 31.4 |
| 65 | 10.0 | 28.6 | 9.5 | 23.5 | 10.8 | 23.0 |
| 67 | 12.8 | 29.4 | 13.0 | 28.2 | 12.5 | 28.0 |
| 68 | 11.9 | 29.6 | 10.9 | 27.9 | 15.1 | 30.1 |
| 69 | 16.0 | 42.0 | 15.5 | 40.8 | 15.0 | 38.8 |
| 70 | 14.6 | 30.8 | 14.8 | 29.9 | 14.7 | 32.4 |
| 71 | 11.6 | 23.8 | 14.5 | 27.6 | 14.9 | 25.4 |
| 72 | 9.8 | 29.0 | 12.9 | 36.1 | 11.5 | 35.3 |
| 73 | 10.5 | 33.3 | 15.2 | 38.1 | 12.9 | 33.2 |
| 74 | 10.0 | 34.3 | 13.6 | 33.6 | 13.7 | 30.3 |
| 76 | 12.3 | 35.8 | 17.3 | 36.0 | 15.2 | 33.4 |
| 78 | 15.6 | 28.1 | 19.1 | 37.1 | 16.6 | 34.2 |
| 79 | 12.5 | 39.7 | 16.2 | 39.5 | 13.9 | 34.5 |
| 80 | 17.3 | 42.5 | 18.1 | 31.6 | 18.1 | 31.7 |
| 81 | 11.1 | 32.1 | 14.8 | 26.4 | 14.5 | 26.8 |
| 82 | 11.1 | 22.7 | 13.0 | 20.5 | 13.8 | 20.2 |
| 83 | 13.9 | 24.3 | 14.4 | 23.8 | 13.6 | 22.3 |
| 84 | 14.4 | 31.0 | 14.2 | 28.1 | 14.1 | 29.3 |
| 85 | 12.3 | 24.6 | 10.9 | 20.0 | 11.8 | 22.0 |
| 86 | 10.8 | 27.2 | 15.8 | 33.5 | 16.4 | 35.2 |
| 87 | 11.1 | 29.7 | 16.1 | 27.1 | 15.7 | 26.1 |
| 88 | 9.3 | 23.6 | 11.0 | 25.0 | 16.8 | 26.0 |
| 89 | 14.6 | 25.9 | 14.0 | 21.0 | 14.0 | 22.6 |
| 90 | 13.7 | 27.1 | 19.0 | 29.4 | 18.0 | 27.9 |

TABLE 22.1
Transepidermal water loss values (E, g m⁻² h⁻¹) for the control

| | | | |
|---|---|---|---|
| Volunteer | D0 | D14 | D28 |

| code | T30 | T0 | T30 | T0 | T30 | T30 |
|---|---|---|---|---|---|---|
| 59 | 13.2 | 41.6 | 16.9 | 40.1 | 15.6 | 39.1 |
| 60 | 9.5 | 22.6 | 15.1 | 29.7 | 12.3 | 27.5 |
| 61 | 13.0 | 26.1 | 13.3 | 28.3 | 16.5 | 31.3 |
| 62 | 8.7 | 17.2 | 9.9 | 19.7 | 10.9 | 21.3 |
| 64 | 17.7 | 35.7 | 19.9 | 32.6 | 20.1 | 32.0 |
| 65 | 10.7 | 27.2 | 13.2 | 28.3 | 14.6 | 30.3 |
| 67 | 13.5 | 27.7 | 14.1 | 31.4 | 15.4 | 31.2 |
| 68 | 10.8 | 30.6 | 13.8 | 31.5 | 18.0 | 33.6 |
| 69 | 17.3 | 41.2 | 17.8 | 42.2 | 17.0 | 42.3 |
| 70 | 16.8 | 34.4 | 18.3 | 33.0 | 18.3 | 34.5 |
| 71 | 10.7 | 20.2 | 14.7 | 26.9 | 10.5 | 22.3 |
| 72 | 10.9 | 27.1 | 14.3 | 36.5 | 12.7 | 32.8 |
| 73 | 11.3 | 35.9 | 10.5 | 36.8 | 17.2 | 42.1 |
| 74 | 12.4 | 39.1 | 15.1 | 35.6 | 13.3 | 35.4 |
| 76 | 12.2 | 33.9 | 18.2 | 37.4 | 17.5 | 34.4 |
| 78 | 16.1 | 29.1 | 22.5 | 41.5 | 17.4 | 35.5 |
| 79 | 10.4 | 35.9 | 14.3 | 37.6 | 15.4 | 36.3 |
| 80 | 15.8 | 38.0 | 17.8 | 37.4 | 15.2 | 35.0 |
| 81 | 11.6 | 30.4 | 13.7 | 30.5 | 14.2 | 27.9 |
| 82 | 11.0 | 24.9 | 11.2 | 21.6 | 15.2 | 24.6 |
| 83 | 14.0 | 26.1 | 16.4 | 27.1 | 15.7 | 27.0 |
| 84 | 13.1 | 31.3 | 16.5 | 33.3 | 16.6 | 35.3 |
| 85 | 9.6 | 24.5 | 12.1 | 24.8 | 13.2 | 27.6 |
| 86 | 10.7 | 25.4 | 15.6 | 30.2 | 14.0 | 28.1 |
| 87 | 13.5 | 31.7 | 15.0 | 32.0 | 19.0 | 33.3 |
| 88 | 11.9 | 28.8 | 10.8 | 22.0 | 15.2 | 29.2 |
| 89 | 13.7 | 27.8 | 15.1 | 28.2 | 14.6 | 30.3 |
| 90 | 15.0 | 25.9 | 15.4 | 24.8 | 15.4 | 25.4 |

TABLE 22.2
ΔE values (Equation 1)

| | D0 | | D14 | | D28 | |
|---|---|---|---|---|---|---|
| Volunteer Code | Product | Control | Product | Control | Product | Control |
| 59 | 25.7 | 28.4 | 18.1 | 23.2 | 18.9 | 23.5 |
| 60 | 14.0 | 13.1 | 9.8 | 14.6 | 10.0 | 15.2 |
| 61 | 13.0 | 13.1 | 9.5 | 15.0 | 10.2 | 14.8 |
| 62 | 8.9 | 8.5 | 8.2 | 9.8 | 8.7 | 10.4 |
| 64 | 15.2 | 18.0 | 13.2 | 12.7 | 13.7 | 11.9 |
| 65 | 18.6 | 16.5 | 14.0 | 15.1 | 12.2 | 15.7 |
| 67 | 16.6 | 14.2 | 15.2 | 17.3 | 15.5 | 15.8 |
| 68 | 17.7 | 19.8 | 17.0 | 17.7 | 15.0 | 15.6 |
| 69 | 26.0 | 23.9 | 25.3 | 24.4 | 23.8 | 25.3 |
| 70 | 16.2 | 17.6 | 15.1 | 14.7 | 17.7 | 16.2 |
| 71 | 12.2 | 9.5 | 13.1 | 12.2 | 10.5 | 11.8 |
| 72 | 19.2 | 16.2 | 23.2 | 22.2 | 23.8 | 20.1 |
| 73 | 22.8 | 24.6 | 22.9 | 26.3 | 20.3 | 24.9 |
| 74 | 24.3 | 26.7 | 20.0 | 20.5 | 16.6 | 22.1 |
| 76 | 23.5 | 21.7 | 18.7 | 19.2 | 18.2 | 16.9 |
| 78 | 12.5 | 13.0 | 18.0 | 19.0 | 17.6 | 18.1 |
| 79 | 27.2 | 25.5 | 23.3 | 23.3 | 20.6 | 20.9 |
| 80 | 25.2 | 22.2 | 13.5 | 19.6 | 13.6 | 19.8 |
| 81 | 21.0 | 18.8 | 11.6 | 16.8 | 12.3 | 13.7 |
| 82 | 11.6 | 13.9 | 7.5 | 10.4 | 6.4 | 9.4 |
| 83 | 10.4 | 12.1 | 9.4 | 10.7 | 8.7 | 11.3 |
| 84 | 16.6 | 18.2 | 13.9 | 16.8 | 15.2 | 18.7 |
| 85 | 12.3 | 14.9 | 9.1 | 12.7 | 10.2 | 14.4 |
| 86 | 16.4 | 14.7 | 17.7 | 14.6 | 18.8 | 14.1 |
| 87 | 18.6 | 18.2 | 11.0 | 17.0 | 10.4 | 14.3 |
| 88 | 14.3 | 16.9 | 14.0 | 11.2 | 9.2 | 14.0 |
| 89 | 11.3 | 14.1 | 7.0 | 13.1 | 8.6 | 15.7 |
| 90 | 13.4 | 10.9 | 10.4 | 9.4 | 9.9 | 10.0 |

TABLE 22.3
RE values (Equation 2)

| | | |
|---|---|---|
| Volunteer | Product | Control |

| code | D14 | D28 | D14 | D28 |
|---|---|---|---|---|
| 59 | 0.70 | 0.74 | 0.82 | 0.83 |
| 60 | 0.70 | 0.71 | 1.11 | 1.16 |
| 61 | 0.73 | 0.78 | 1.15 | 1.13 |
| 62 | 0.92 | 0.98 | 1.15 | 1.22 |
| 64 | 0.87 | 0.90 | 0.71 | 0.66 |
| 65 | 0.75 | 0.66 | 0.92 | 0.95 |
| 67 | 0.92 | 0.93 | 1.22 | 1.11 |
| 68 | 0.96 | 0.85 | 0.89 | 0.79 |
| 69 | 0.97 | 0.92 | 1.02 | 1.06 |
| 70 | 0.93 | 1.09 | 0.84 | 0.92 |
| 71 | 1.07 | 0.86 | 1.28 | 1.24 |
| 72 | 1.21 | 1.24 | 1.37 | 1.24 |
| 73 | 1.00 | 0.89 | 1.07 | 1.01 |
| 74 | 0.82 | 0.68 | 0.77 | 0.83 |
| 76 | 0.80 | 0.77 | 0.88 | 0.78 |
| 78 | 1.44 | 1.41 | 1.46 | 1.39 |
| 79 | 0.86 | 0.76 | 0.91 | 0.82 |
| 80 | 0.54 | 0.54 | 0.88 | 0.89 |
| 81 | 0.55 | 0.59 | 0.89 | 0.73 |
| 82 | 0.65 | 0.55 | 0.75 | 0.68 |
| 83 | 0.90 | 0.84 | 0.88 | 0.93 |
| 84 | 0.84 | 0.92 | 0.92 | 1.03 |
| 85 | 0.74 | 0.83 | 0.85 | 0.97 |
| 86 | 1.08 | 1.15 | 0.99 | 0.96 |
| 87 | 0.59 | 0.56 | 0.93 | 0.79 |
| 88 | 0.98 | 0.64 | 0.66 | 0.83 |
| 89 | 0.62 | 0.76 | 0.93 | 1.11 |
| 90 | 0.78 | 0.74 | 0.86 | 0.92 |

TABLE 22.4
FB values (Equation 3)

| Volunteer code | D14 | D28 |
|---|---|---|
| 59 | 11.3 | 9.21 |
| 60 | 41.5 | 44.60 |
| 61 | 41.4 | 34.52 |
| 62 | 23.2 | 24.60 |
| 64 | −16.3 | −24.02 |
| 65 | 16.2 | 29.56 |
| 67 | 30.3 | 17.89 |
| 68 | −6.7 | −5.96 |
| 69 | 4.8 | 14.32 |
| 70 | −9.7 | −17.21 |
| 71 | 21.0 | 38.14 |
| 72 | 16.2 | 0.12 |
| 73 | 6.5 | 12.18 |
| 74 | −5.5 | 14.46 |
| 76 | 8.9 | 0.43 |
| 78 | 2.2 | −1.57 |
| 79 | 5.7 | 6.23 |
| 80 | 34.7 | 35.22 |
| 81 | 34.1 | 14.30 |
| 82 | 10.2 | 12.45 |
| 83 | −2.0 | 9.73 |
| 84 | 8.6 | 11.18 |
| 85 | 11.3 | 13.72 |
| 86 | −8.6 | −18.72 |
| 87 | 34.3 | 22.66 |
| 88 | −31.6 | 18.50 |
| 89 | 31.0 | 35.24 |
| 90 | 8.6 | 17.86 |

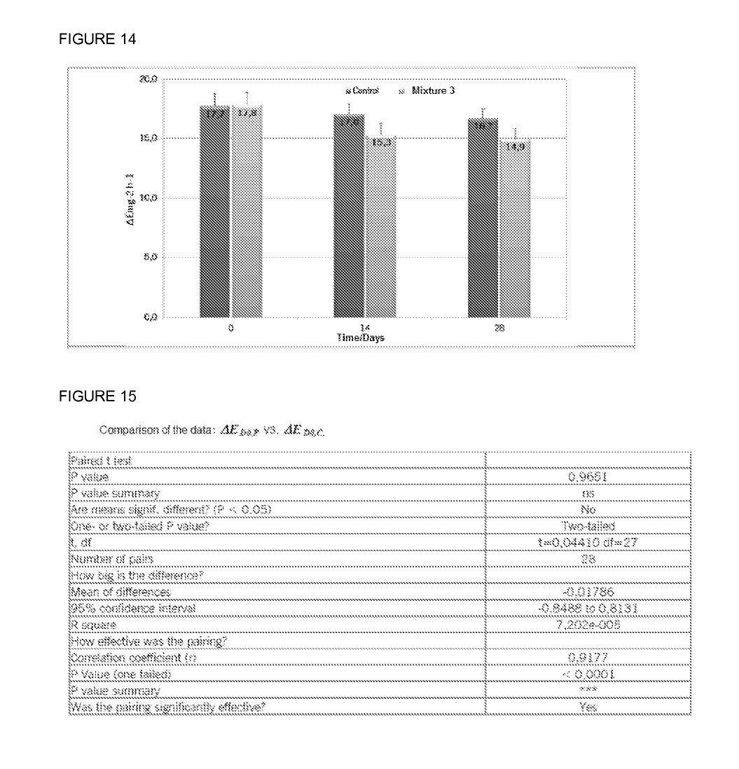

FIG. 14 illustrates the averages of the variation in transepidermal water loss (ΔEDi) according to time (i=0.14 or 28 days) for the forearm wherein the product was applied and for the respective control forearm.

5.4.1. Basal Homogeneity

Table 23 summarizes the results obtained from the statistical analysis of basal data homogeneity (FIG. 15).

TABLE 23
Summarized data on the statistical analysis. P values

| Comparison Group: | Parameter: D0 |
|---|---|
| ΔE_{Product} vs. ΔE_{Control} | 0.9651 (non-significant) |

According to the obtained results there was no statistically significant difference (P>0.05) among the basal values of variation in transepidermal water loss between the forearms wherein the product was applied and the respective control forearms.

5.4.2. Significance of the Effect

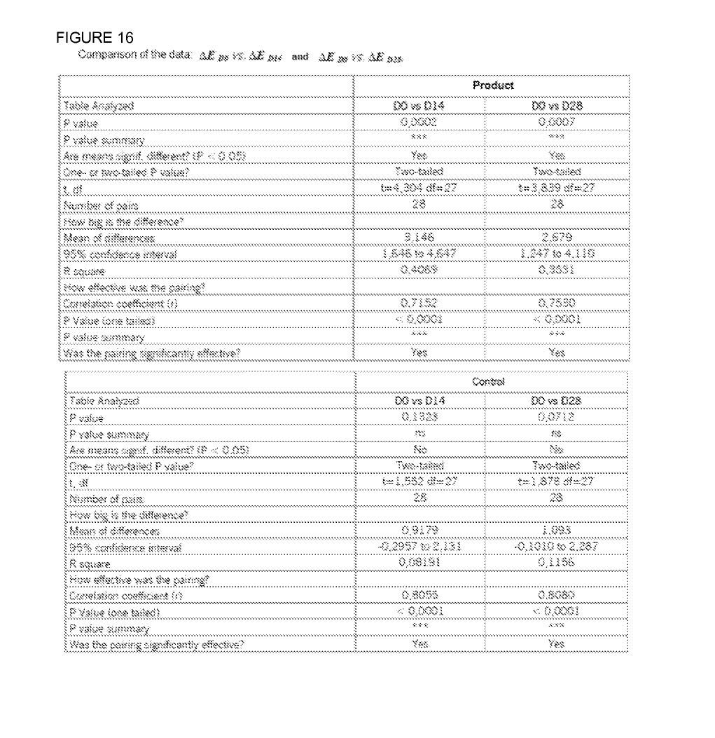

Table 24 shows the Results obtained from the statistical evaluation of the significance of variations in the transepidermal water loss values throughout the study (FIG. 16).

TABLE 24
Summarized data of the statistical analysis of the significance of
the changes in the skin barrier. P values

| Comparison group | Control | Mixture 2 |
|---|---|---|
| ΔE D0 vs. ΔE D14 | 0.1323 (non-significant) | 0.0002 (significant) |
| ΔE D0 vs. ΔE D28 | 0.0712 (non-significant) | <0.0007 (significant) |

According to the results obtained for the control, there were observed no significant variations in the TEWL values after 14 and 28 days, indicating that there were no significant changes in the skin barrier throughout the study.

For MIXTURE 3, a significant reduction in the TEWL variation values after 14 and 28 days of continuous use was noticed, which indicates that the use of said product resulted in significant changes in the skin barrier with respect to the strengthening.

5.4.3. Comparison Between Product and Control

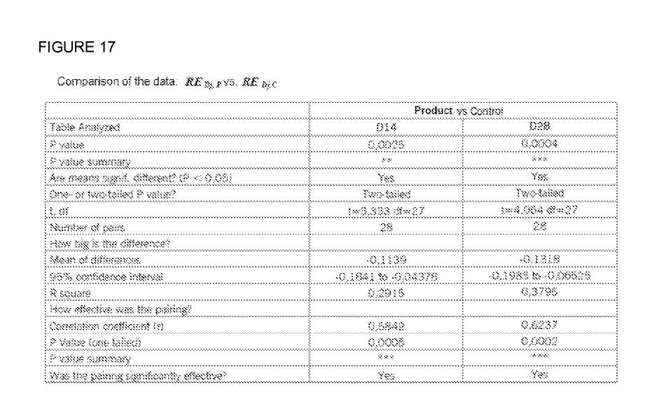

The results of the statistical analysis for assessment of the significance of the effect of the product in relation to the control are displayed in Table 25 (FIG. 17).

TABLE 25
Statistical analysis: product vs. control. P values

| Comparison group | D14 | D28 |
|---|---|---|
| RE_{P} vs. RE_{C} | 0.0025 (significant) | <0.0004 (significant) |

According to the obtained results, after 14 and 28 days of continuous use of MIXTURE 3, the observed reduction of the TEWL values was significantly superior to that observed for the control, indicating that the use of the product provided a significant strengthening effect on the skin barrier already after 14 days of use.

MIXTURE 3 provided a significant strengthening effect on the skin barrier (FB), in relation to the initial skin condition and to the control, of 11% after 14 days of use and 13% after 28 days of use.

5.5. Analysis of Daily Use

89% of the volunteers used the product correctly.

11% of the volunteers stopped using the product for 1 or 2 days, stating they had forgotten about it.

### 6. Conclusion

According to the study conditions disclosed in this report, it can be concluded that MIXTURE 3 applied to the skin in the region of the forearm significantly strengthened the skin barrier when compared to the control (skin free of any products) after 14 and 28 days of continuous use.

The percentage value obtained for the strengthening of skin barrier compared to the initial state of the skin and to the control was of 11% after 14 days of use and 13% after 28 days of use.

### TEST 5—Evaluation of Film Formation on VitroSkin® (Artificial Skin) by Optical Microscopy

In order to develop excellent products, quantitative and qualitative techniques are needed for characterizing and knowing the effects, and subsequently characterizing the products for each type of application.

There are numerous the professional industrial activities, handcraft activities, activities in hospitals, medical offices and household cleaning which are associated with frequent hand washing, with the intense contact of the skin with highly irritating and allergenic substances (e.g.: metallic salt compounds, lubricants, coolants, detergents, disinfectants, shampoos, among others). The often simultaneous influence of these factors may consequently lead to the phenomenon of dryness and loss of the natural skin barrier especially in people with dry and sensitive skin, preferably in the hands, in the form of cumulative-toxic eczemas and contact-allergic eczemas.

The water used in the skin cleansing process eliminates a considerable amount of natural moisturizing substances located in the cells of the stratum corneum. The surfactants and other components of the cleansing products may cause a pronounced removal of the oily layer and a consequent disorder of the epidermal barrier function. This process causes an increased loss of transepidermical water, so that the skin feels dry and rough.

The stratum corneum forms a closed barrier between the body and the environment, preventing it from drying out and protecting it from environmental influences. It is formed by corneocytes snd lipids connected by protein structures which act as a kind of cellular concrete. The lipid layers are formed by fatty acids, cholesterol, triglycerides and ceramides.

This removal of the protective barrier, also called wear dermatosis, facilitates the opening of paths and free areas for the penetration and diffusion of surfactants and other irritant agents which induce the allergic-contact potential, penetratete into the deepest layers more easily, thus triggering pathological mechanisms of a cumulative-toxic eczema or a contact-allergic eczema.

Mechanisms of action of surfactant components on skin:

1. Active surface properties;

2. Elimination of substances of the own skin, of the hydrolipidic layer and of the horny layer;

3. Skin dehydration;

4. Limited barrier function;

5. Easy penetration of surfactant molecules into the deepest skin layers;

6. Induction of inflammatory mechanisms;

7. Formation of cumulative-toxic eczema.

The evaluation of the effect of a cosmetic treatment on skin may be performed by the analysis of surface properties, especially topography.

### 1. Objective

Evaluate the strengthening effect on the skin barrier caused by the home use of MIXTURE 3 after 14 and 28 days by using a process for removing layers of the stratum corneum with 30 successive applications and removals of the adhesive tape and evaluate the transepidermal water loss by evaporimetry.

The present study aimed at evaluating the effectiveness of MIXTURE 1, MIXTURE 2 and MIXTURE 3 applied to the skin as to their film formation property.

The study was performed in vitro using Vitroskin® (artificial skin) as substrate and the measurements were made by the optical microscopy technique.

### 2. Procedures

2.1. Preparation of Vitro-Skin®

For each sample there were prepared 6 Vitro-Skin substrates measuring 3.0 cm×2.5 cm, which were placed in plastic slides for fixation, provided by IMS-USA, conferring a free area for product application of 5.0 cm². Prior to use, the substrates were kept for 16 hours in a hydration chamber (IMS-USA) containing a solution of 15% (w/w) of glycerine in water.

2.2. Standard Application of the Product on the Substrate

Control Group: It was applied on 5 previously hydrated substrates 10 μL of destilled water, massaging them with a finger stall for 30 seconds with circular motion, and then drying them in an oven at 30° C. for 1 hour.

Product Group: It was applied on 5 previously hydrated substrates 10 μL of the product, massaging them with a finger stall for 30 seconds with circular motion, and then drying them in an oven at 30° C. for 1 hour.

2.3. Evaluation by Optical Microscopy

Optical microscopy was performed using the Olympus BX53 microscope using 10× objective. Three regions were analyzed for each substrate, making a total of 15 regions for each study group.

3. Software for acquisition and analysis of data

CellSens® 1.4 (Olympus Corporation, Japan);

Scion Image for Windows (Scion Corp, NIH, USA);

Microsoft® Office Excel 2010 (Microsoft, USA);

GraphPad Prism 6 (GraphPad, California, USA).

### 4. Results and Discussion

Vitro-Skin® was used as substrate, provided by IMS Inc (USA). According to the manufacturer, Vitro Skin contains the protein and lipid components of the skin, mimicking the topography, pH, critical surface tension and ionic strength of the human skin (http://www.ims-usa.com).

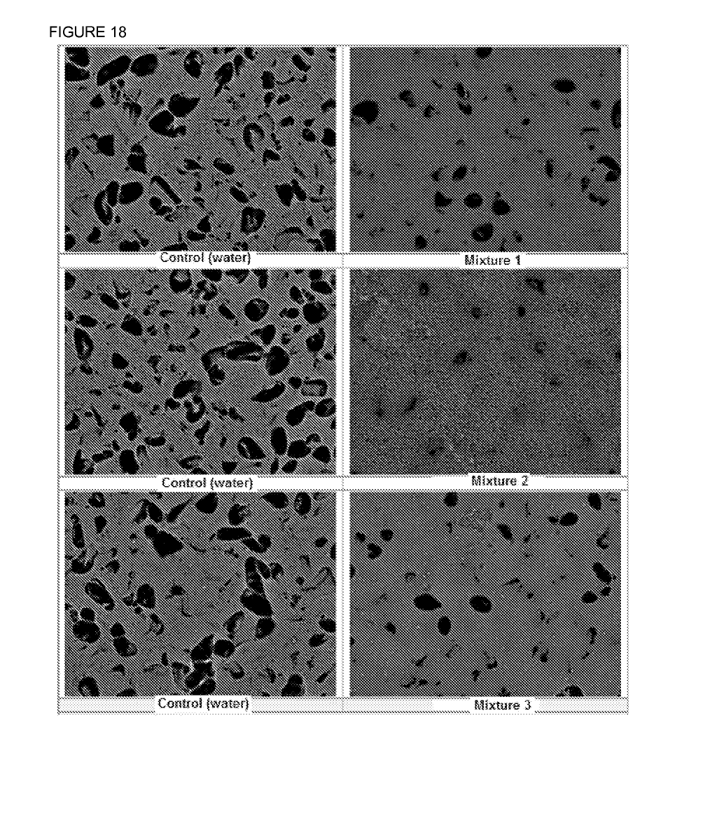

FIG. 18 illustrates a set of optical microscopy images obtained for the samples analyzed, applied under the substrate-Vitro Skin®, compared with the control group, in which the application consisted of distilled water.

The deposition of materials on the rough surface of Vitro-Skin® (RMS=80 μm) alters the phase contrast observed in the optical microscopy. The formation of light and dark regions in the images of optical microscopy depends on the physicochemical characteristics of the depositing material. Substrates treated with the mixture under study showed the formation of dark areas, related to the deposited material.

Through the analysis of images, the images obtained are binarized, and the region concerning the deposited material is identified as black pixels.

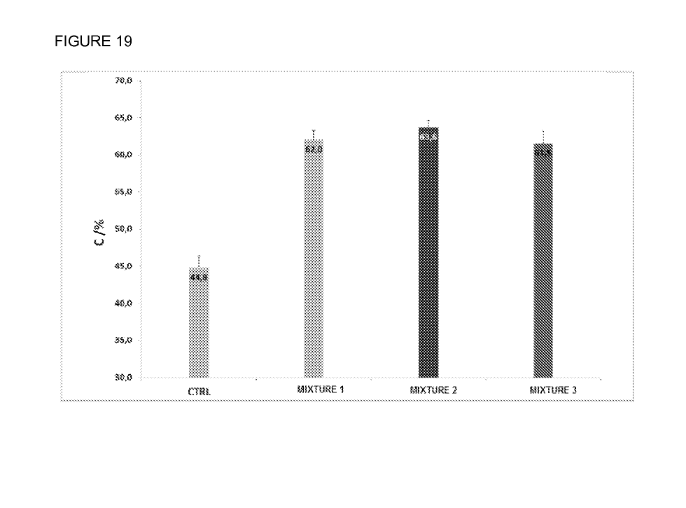

The images were binarized by adjusting the histogram of 8-bits for the range 0-130, determining the percentage of black pixels (C). The higher the percentage of black pixels, greater the amount of material deposited on the surface of Vitro-Skin®. Table 26 shows the values of C (black pixel count, %) for each image analyzed. FIG. 19 illustrates the average results of these values.

TABLE 26
Values of C (counting black pixels, %) of each image analyzed.

| Image | | | | |
|---|---|---|---|---|
| number | Control | MIXTURE 1 | MIXTURE 2 | MIXTURE 3 |
| 1 | 43.2 | 62.3 | 63.4 | 63.4 |
| 2 | 45.3 | 63.0 | 62.0 | 60.8 |
| 3 | 46.6 | 63.2 | 62.8 | 58.9 |
| 4 | 43.8 | 62.5 | 63.2 | 62.3 |
| 5 | 42.4 | 61.6 | 64.2 | 61.9 |
| 6 | 45.1 | 63.0 | 63.4 | 60.1 |
| 7 | 48.3 | 60.5 | 62.8 | 62.4 |
| 8 | 43.6 | 59.9 | 63.4 | 63.4 |
| 9 | 45.9 | 63.9 | 65.2 | 63.2 |
| 10 | 44.0 | 60.3 | 63.3 | 64.7 |
| 11 | 43.7 | 61.2 | 65.0 | 60.6 |
| 12 | 43.6 | 60.7 | 65.1 | 60.1 |
| 13 | 45.8 | 61.5 | 64.6 | 60.2 |
| 14 | 46.9 | 64.1 | 63.2 | 60.7 |
| 15 | 43.2 | 62.1 | 63.2 | 59.4 |

Data obtained were analyzed using the method of analysis of single factor variance, with Tukey multiple comparison post-test, considering a confidence interval of 95%.

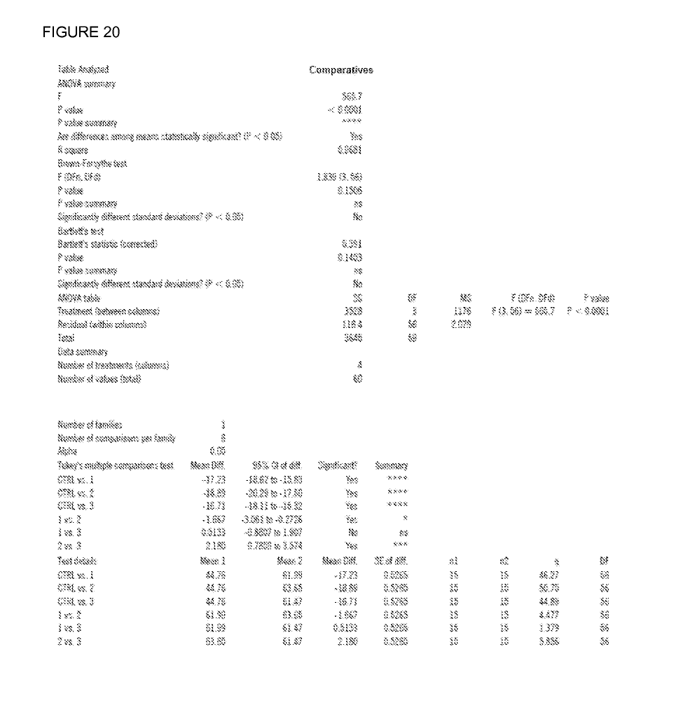

According to the statistical results obtained in FIG. 20, all samples showed significant film formation compared to the control.

MIXTURE 2 showed film forming results significantly better than MIXTURE 1 and MIXTURE 3. There was no statistical difference between MIXTURE 1 and MIXTURE 3.

### 5. Conclusion

According to the results of the study, it was found that the application of the samples of MIXTURE 1, MIXTURE 2 and MIXTURE 3 on the artificial skin (Vitro Skin®) provided significantly higher film formation than the control (Vitro-Skin® treated with water only).

Thus, it can be concluded that MIXTURE 1, MIXTURE 2 and MIXTURE 3 are capable of forming a film on human skin.

The sample MIXTURE 2 showed film forming results significantly better than MIXTURE 1 and MIXTURE 3. There was no statistical difference between MIXTURE 1 and MIXTURE 3.

### TEST 6—Evaluation of the Nail Strengthening Effect

### 1. Objective

This study aims to assess the potential for strengthening nails due to the use of cosmetics.

### 2. Experimental Design

The nail strengthening study was carried out in-vitro on a synthetic nail mimicking the human nail, Vitro-Nails®, manufactured by IMS-USA.

Vitro-Nails® contains lipidic and protein components, mimicking the wetting, thickness and flexibility properties of human nails (Sottery, J. P.; Jaramillo, J. H. A New Substrate for the Rapid, In Vitro Assessment of Nail Care Products. IMS Inc, Society of Cosmetic Chemists Annual Scientific Metting, 1998).

Thirty Vitro-Nails® plates cut into 2.8×7.0 cm size were used. Divided into three treatment groups (mixtures):

MIXTURE 1: code T01

MIXTURE 2: code T02

MIXTURE 3: code T03

The plates underwent a pre-cleansing procedure in which a paper towel soaked with a nail polish remover was used.

After cleaning, the basal measurements (initial) of the bending strength of the plates were obtained using a universal testing machine EMIC DL500, with a load cell of 20N.

After obtaining the basal measurements, 50 mg of the products were applied in each plate. The products were spread over the entire surface (of one side of the plate) for 1 minute. After applying the product, the plates were kept in an incubator at 36° C. for 15 minutes to dry and new bending strength measurements were carried out—final measurements (Final).

### 3. Bending Strength Measurements

Nail strengthening is related to the strength required to bend the nail. Through the stress-strain curves obtained in the study, the necessary force values in Newton (N) necessary to vertically bend the nail in 1.0 mm were obtained.

For the bending of the plates, a suitable support was used with free horizontal range of 35 mm and EMIC universal testing machine, provided with 20N load cell. Descending speed of the probe tip used was 10 mm/min.

### 4. Results and Discussion

The bending strength results obtained for the study groups evaluated are shown in Table 27:

TABLE 27
Experimental Data Obtained
Force Values (N) obtained for 1.0 mm deformation

| | |
|---|---|
| Initial | Final |

Mixture 1

| | |
|---|---|
| 1.32 | 2.28 |
| 0.90 | 1.68 |
| 1.08 | 1.92 |
| 0.78 | 1.32 |
| 0.84 | 1.50 |
| 1.20 | 2.10 |
| 0.72 | 1.20 |
| 0.96 | 1.80 |
| 0.84 | 1.44 |
| 0.78 | 1.38 |

Mixture 2

| | |
|---|---|
| 1.08 | 1.86 |
| 0.84 | 1.38 |
| 0.90 | 1.50 |
| 1.26 | 2.28 |
| 1.38 | 2.34 |
| 1.02 | 1.80 |
| 0.78 | 1.20 |
| 0.84 | 1.38 |
| 1.20 | 2.28 |
| 0.90 | 1.56 |

Mixture 3

| | |
|---|---|
| 1.20 | 1.39 |
| 0.96 | 1.19 |
| 1.26 | 1.32 |
| 0.72 | 0.92 |
| 0.78 | 0.99 |
| 0.72 | 0.99 |
| 0.66 | 0.92 |
| 1.38 | 1.12 |
| 0.78 | 0.99 |
| 0.96 | 1.12 |

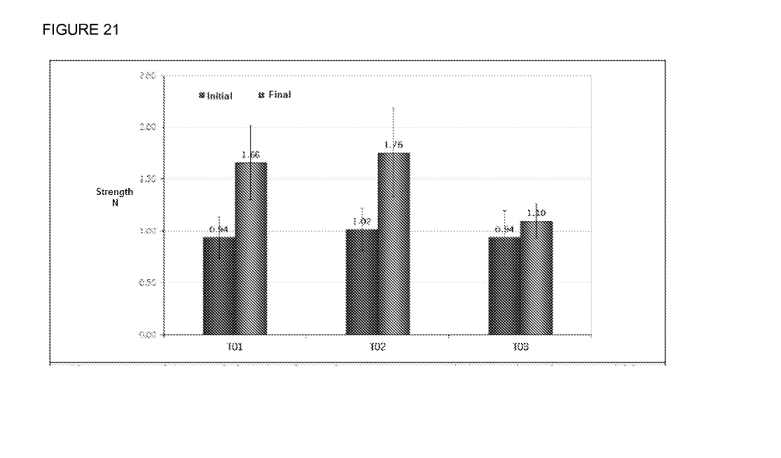

FIG. 21 displays the average results obtained: Average bending strength values in (N) of the Vitro-Nails® plates before (initial) and after cosmetic treatment (final) for each group of study. Mean±SD.

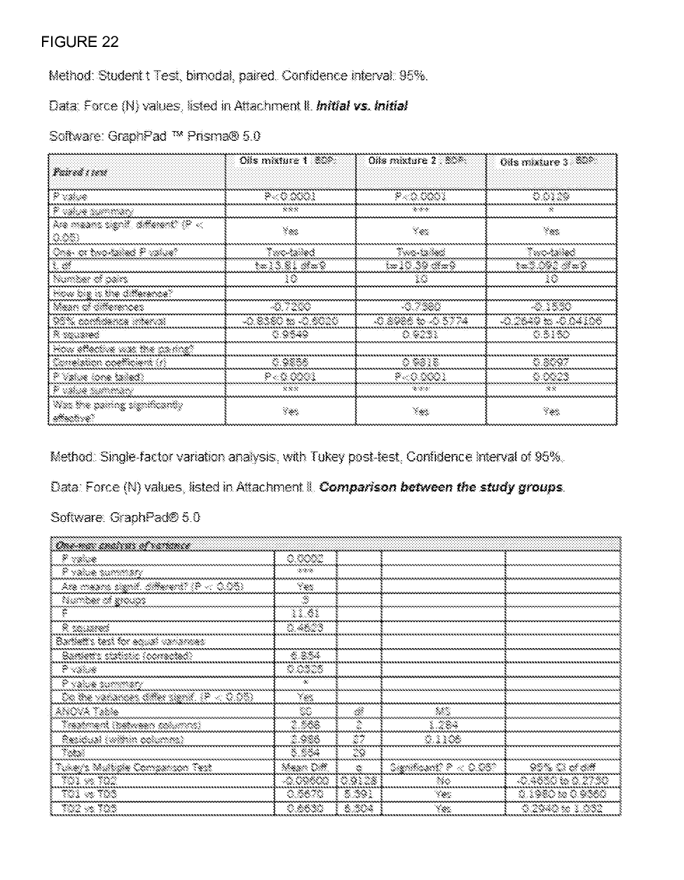

The data obtained for bending strength, initial and final, were statistically compared by the paired, bimodal Student's t-Test method, considering a confidence interval of 95%. Table 28 summarizes the results obtained, listed in FIG. 22.

TABLE 28
Results of statistical analysis. Initial vs. Final. P values for I.C. of 95%

| Compared Grupos | P value | Did it show significant differences? |
|---|---|---|
| T01 | P < 0.05 | Yes |
| T02 | P < 0.05 | Yes |
| T03 | P < 0.05 | Yes |

According to the results obtained, the Vitro-Nails® plates subjected to the application of the products: Mixture 1, Mixture 2 and Mixture 3, showed higher bending strength compared with the plates of Vitro-Nails® without treatments (initial state).

Table 29 illustrates the “potential nail strengthening, (PF)” in percentage and number of times, calculated in relation to the initial state, according to Equations 1 and 2.

$${P}_{F} ( \% ) = 100 * ( \frac{{F}_{final} - {F}_{initial}}{{F}_{initial}} )$$

Equation 1: Calculation of the nail strengthening potential (%) of the treatment in relation to the initial state, wherein: Fᵢₙᵢₜᵢₐₗ=Force values for the initial state; F_{final}=Force values for the final state.

$${P}_{F} = \frac{{F}_{final}}{{F}_{initial}}$$

Equation 2: Calculation of the nail strengthening potential (in number of times) of the treatment in relation to the initial state, wherein: Fᵢₙᵢₜᵢₐₗ=Force values for the initial state; F_{final}=Force values for the final state.

TABLE 29
Nail strengthening potential after cosmetic treatment in relation to
the initial state (without treatment).

| Treatment | % | Number of times |
|---|---|---|
| Mixture 1 | 76 | 1.8 |
| Mixture 2 | 72 | 1.7 |
| Mixture 3 | 16 | 1.2 |

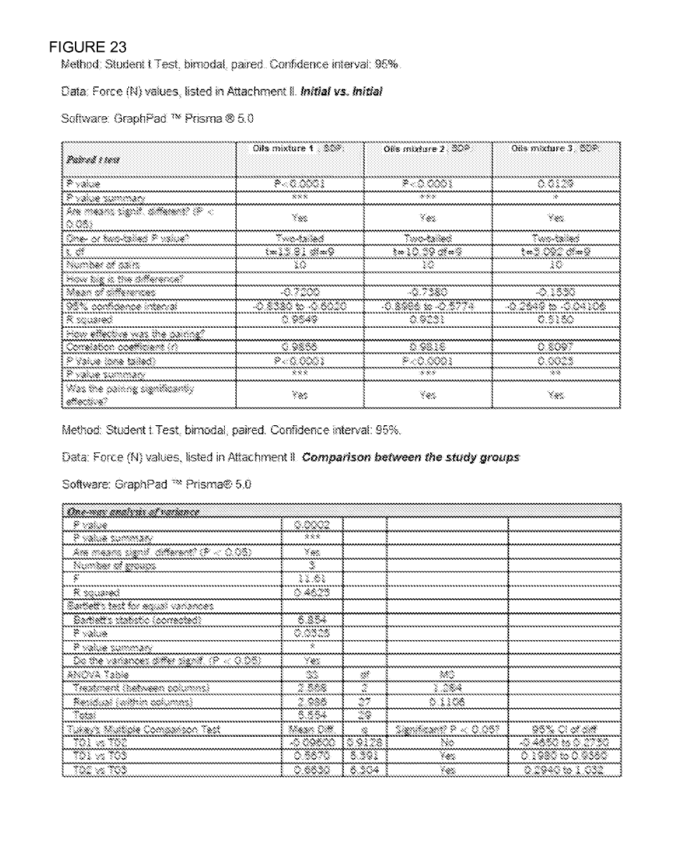

The comparison between treatments was performed using the method of analysis of single factor variance, with Tukey multiple comparison post-test, considering a confidence interval of 95%. The complete results of the statistical analysis are shown in FIG. 23.

According to the results obtained there was no statistically significant difference between the bending strength final values between groups T01 and T02.

The treatment group T03 had lower bending strength compared to the treatment groups T01 and T02.

### 5. Conclusion

In the present study, Vitro-Nails® synthetic nail plates underwent cosmetic treatment with the following products:

Mixture 1

Mixture 2

Mixture 3

According to the results obtained from the stress-strain testing, the application of the products:

Mixture 1, Mixture 2 and Mixture 3 on synthetic nails significantly increased the force required for bending the same.

Thus it can be said that the treatments with the products: Mixture 1, Mixture 2 and Mixture 3 promote significant nail strengthening.

Table 30 below represents the nail strengthening potential of the study groups:

TABLE 30
Nail strengthening potential after cosmetic treatment in relation to
the basal state (without treatment)

| Treatment | % | Number of times |
|---|---|---|
| Mixture 1 | 76 | 1.8 |
| Mixture 2 | 72 | 1.7 |
| Mixture 3 | 16 | 1.2 |

Still according to the results obtained there was no statistically significant difference between the bending strength final values between the Products Mixture 1, and Mixture 2.

The treatment group Mixture 3 presented lower bending strength compared to treatment groups Mixture 1 and Mixture 2.

Whereas according to IMS-USA (www.ims-usa.with) the substrate Vitro-Nail® is regarded as a material that mimics the human nail, we infer that the properties obtained from this “in vitro” study can be extrapolated to the human nail.

### TEST 7—Study of the Restructuring Potential

In this study, the scanning electron microscopy (SEM) technique was used to evaluate the surface of hair fibers subjected to different dyes and products for hair treatment. The technique allows obtaining high resolution images of the fiber previously coated with a conductive layer of gold. The image results from the secondary and backscattered electrons formed during scanning the sample surface with a high intensity electron beam.

In conjunction with image analysis softwares it is possible to standardize the images and quantify their surface damage, allowing a more accurate comparison of the action of different products for hair care.

### 1. Objective

This study aims to evaluate by scanning electron microscopy, combined with image analysis, the levels of damage to the surface of the hair fiber subjected to cosmetic treatments.

### 2. Experimental Design

Twelve (12) locks of caucasian bleached hair were prepared (DeMeoBrothers INC, NY-USA), weighing 5.0 g and measuring 25 cm. All locks of hair were subjected to a pre-cleaning standardized process using a 10% solution of Sodium Lauryl Ether Sulfate (SLES) for 1 minute followed by rinsing in running water. The locks were dried in a standardized environment at 55±5% relative humidity and 22±2° C., for 24 hours before the assays.

The twelve (12) locks were subsequently divided into 4 groups:

TABLE 31

| Treatments | Code in the study |
|---|---|
| SLES10% | CTRL |
| Mixture 1 | T01 |
| Mixture 2 | T02 |
| Mixture 3 | T03 |

Strands of hair were removed for analysis by SEM after 24 hours of drying in a controlled environment at 55±5% relative humidity and 22±2° C.

The procedures for application of the products and SEM analysis are described below:

A. Preparation of the Samples

Twelve (12) locks of Caucasian bleached hair were prepared (DeMeoBrothers INC, NY-USA), weighing 5.0 g and measuring 25 cm. All locks of hair were subjected to a pre-cleaning standardized process using a 10% solution of Sodium Lauryl Ether Sulfate (SLES) for 1 minute followed by rinsing in running water. The locks were dried in a standardized environment at 55±5% relative humidity and 22±2° C., for 24 hours before the assays.

A.1. CTRL Group

a) Wet the lock for 20 s and remove the excess water.

b) Apply 1.0 mL of 10% SLES and massage for 60 seconds. Rinse locks for 60 seconds to remove excess water.

A.2. Groups T01, T02 and T03

a) Water should be at 35-40° C.

b) Wet the lock for 20 s and remove the excess water.

c) Apply 0.5 g of the mixture and massage for 60 seconds. Do not rinse.

B. Scanning Electron Microscopy.

From the collected strands, segments with 5 mm length were removed from the central region. These segments were fixed to the sample holder (previously identified in accordance with the group of locks) of the microscope, using carbon-based conductive tape.

The samples were coated with a 90 Å gold conductive layer using the equipment Spulter Balzers SCD-050 Coater. For each treatment group 5 micrographs were acquired randomly, using the scanning electron microscope ZEISS™ 940-A, at 15 kV.

C. Image Analysis.

The micrographs obtained were subjected to the image analysis software Scion® for Windows, to quantify the observed morphological differences.

To this end, an algorithm was used which was developed to standardize the images with respect to the brightness, contrast and intensity parameters, to detect and quantify the lighter areas of the image as a percentage of white Pixels, correlated with elevations on the surface, such as fragments and edges of raised cuticles.

### 3. Results and Discussions

From the photomicrographs obtained, we proceeded to the analysis of images using the software Scion® for Windows, in order to detect damage on the fiber surface (raised cuticle, fragments) characterized by lighter regions.

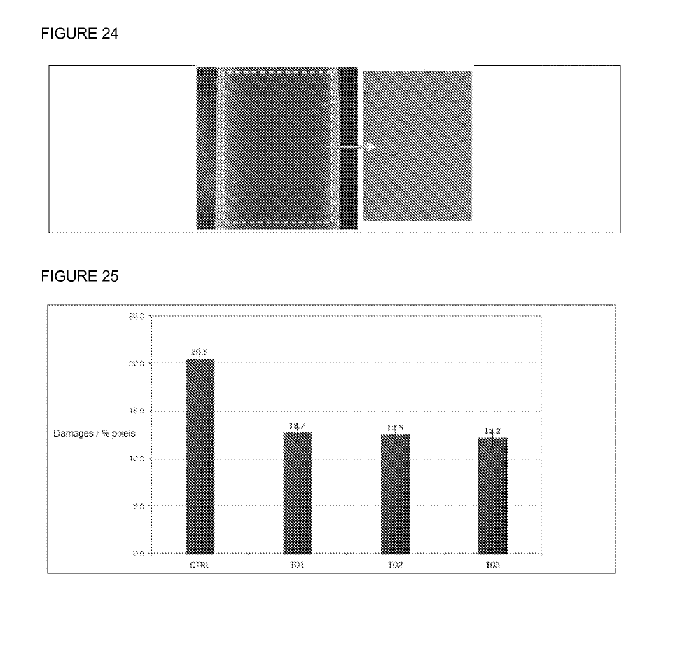

To quantify the level of damage on the fiber surface, the parameter “Percent Damage, D” was determined. This parameter represents the percentage of white pixels in relation to the total number of pixels (kept constant) of the converted images into a white:black binary system—FIG. 24.

Table 32 shows the results of image analysis for each sample, from which FIG. 25 was obtained.

TABLE 32
Experimental Data Obtained through Image Analysis Software:
Scion ® for Windows and Damage % Values obtained through
software Scion ® for Windows

| CTRL | T01 | T02 | T03 |
|---|---|---|---|
| 19.14 | 11.55 | 12.71 | 11.75 |
| 21.64 | 13.20 | 13.39 | 10.88 |
| 20.66 | 11.99 | 11.87 | 12.76 |
| 21.20 | 13.70 | 13.25 | 13.09 |
| 19.72 | 13.13 | 11.39 | 12.32 |

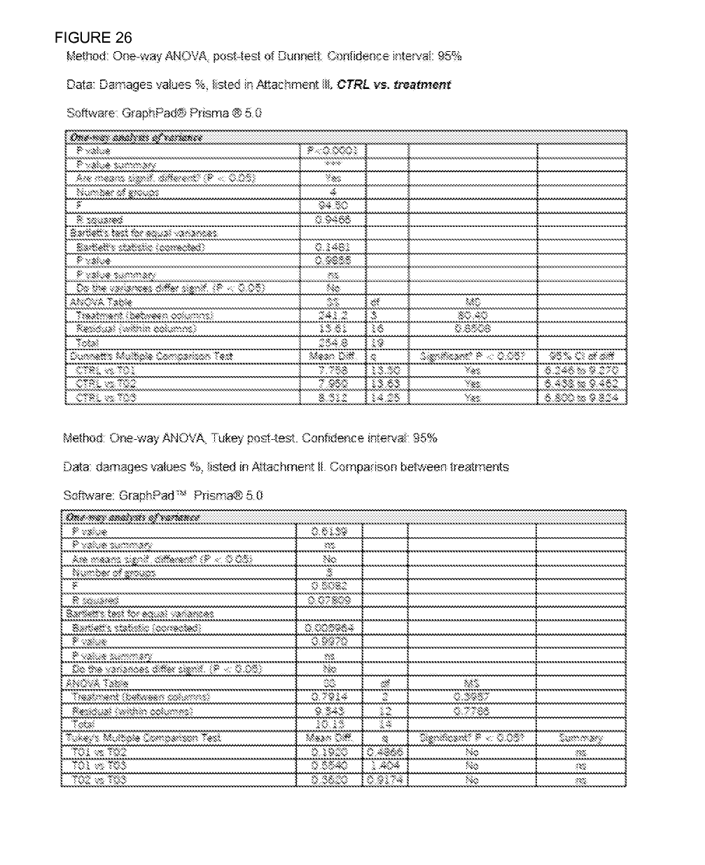

The results obtained for the treatments were statistically compared with the CTRL group using the method of analysis of single factor variance, with Dunnett multiple comparison post-test, considering a confidence interval of 95%. The results obtained are listed in FIG. 26.

According to the obtained results, treatments T01, T02 and T03 showed significantly lower damage values in relation to the CTRL group.

For the comparison between treatments, the results obtained for the groups T01, T02 and T03 were analyzed by the single factor variance method, with Tukey multiple comparison post-test, considering a confidence interval of 95%. The results obtained are listed in FIG. 26.

According to the results obtained there was no statistically significant difference on the Damage values between groups T01, T02 and T03.

Table 33 illustrates the “Damage Reduction, RD” in percentage and number of times, calculated relative to the CTRL group, according to Equations 1 and 2.

$$RD ( \% ) = 100 * ( \frac{{D}_{CTRL} - {D}_{TRAT}}{{D}_{CTRL}} )$$

Equation 1: Calculation of Damage Reduction (%) of the treatments compared to the CTRL group, wherein: DCTRL=Damage values for the CTRL group; DTRAT=Damage values for the study groups

$$RD = \frac{{D}_{CTRL}}{{D}_{TRAT}}$$

Equation 2: Calculation of Damage Reduction (in number of times) of the treatments in relation to the CTRL group, wherein: DCTRL=Damage values for the CTRL group; DTRAT=Damage values for the study groups

TABLE 33
Damage Reduction (% and number of times) of the treatment
groups in relation to the CTRL group

| Treatment | % | Number of times |
|---|---|---|
| T01 | 38 | 1.6 |
| T02 | 39 | 1.6 |
| T03 | 41 | 1.7 |

### 4. Conclusions

The reduction in damage, determined by means of the combined scanning electron microscopy image analysis, consists of an improved cuticular surface, seating the edges of the cuticle and removing fragments.

In the present study, locks of bleached caucasian hair were subjected to treatments with the following product:

SLES 10% —CTRL group;

Mixture 1—group T01;

Mixture 2—group T02;

Mixture 3—group T03;

According to the results, the locks subjected to treatment T01 exhibited 38% (or 1.6 times) less surface damage in relation to the locks subjected to treatment with SLES 10%.

The locks subjected to treatment T02 exhibited 39% (or 1.6 times) less surface damage in relation to the locks subjected to treatment with SLES 10%.

The locks subjected to treatment T03 exhibited 41% (or 1.7 times) less surface damage in relation to the locks subjected to treatment with SLES 10%.

There was no difference in the surface damage of hair subjected to treatments T01, T02 and T03.

### TEST 8—Research on Human Hair Substantiveness by Fluorescence Microscopy

Measurements of the substantiveness of chemical components present in cosmetic formulations can be made by various methods, but none is as accurate as using the technique of component marking with fluorescent molecules using fluorescence microscopy. This technique allows discriminating the components present on the surface and inside the hair fiber.

Experimentally, it is extremely difficult to detect the microscopic distribution of small quantities of substances within a similar chemical composition, for example amino acids. An essential requirement is the treatment process and selection of the fluorescent component; the advantage of using this specific technique is the high specificity of the selection through the fluorescent emission of the marker.

In this study, the dye Rhodamine B (CI Basic Violet 10) a cationic dye, which reacts with the active sites of the sulfonic acid formed from the cleavage of the S—S bond of cystine (disulfide bonds) caused in the hair relaxing process. A fluorescent complex is formed in the hair fiber, which is detected when exposed to fluorescence microscope attached to compatible filters to the wavelength emitted.

### 1. Objective

This study aims to assess the level of superficial and internal substantiveness of cosmetic prototypes in fibers of human hair, by fluorescence microscopy analysis.

### 2. Experimental Design

Twelve (12) locks of Caucasian bleached hair were prepared (DeMeoBrothers INC, NY-USA), weighing 5.0 g and measuring 25 cm. All locks of hair were subjected to a pre-cleaning standardized process using a 10% solution of Sodium Lauryl Ether Sulfate (SLES 10%) for 1 minute followed by rinsing in running water. The locks were dried in a standardized environment at 55±5% relative humidity and 22±2° C., for 24 hours before the assays.

The twelve (12) locks were divided into 04 treatment groups, as illustrated in Table 34:

TABLE 34

| Treatments | Code in the study |
|---|---|
| SLES10% | CTRL |
| MIXTURE 1 | T01 |
| | Tael |
| MIXTURE 2 | T02 |
| MIXTURE 3 | T03 |

The procedures for application of the products are described below:

A. Preparation of the Samples

Twelve (12) locks of Caucasian bleached hair were prepared (DeMeoBrothers INC, NY-USA), weighing 5.0 g and measuring 25 cm. All locks of hair were subjected to a pre-cleaning standardized process using a 10% solution of Sodium Lauryl Ether Sulfate (SLES) for 1 minute followed by rinsing in running water. The locks were dried in a standardized environment at 55±5% relative humidity and 22±2° C., for 24 hours before the assays.

B. Treatment of the Locks

B.1. CTRL Group
a) Wet the lock for 20 s and remove the excess water.

b) Apply 1.0 mL of 10% SLES and massage for 60 seconds. Rinse locks for 60 seconds to remove excess water.

B.2. Groups T01, T02 and T03
a) Water should be at 35-40° C.
b) Wet the lock for 20 s and remove the excess water.
c) Apply 0.5 g of the mixture and massage for 60 seconds. Do not rinse.

After the treatments, the locks of hair were dried for 24 hours in a controlled environment at 55±5% relative humidity and 22±2° C. Then strands of hair were randomly removed from the locks. The strands were analyzed by fluorescence microscopy.

### 3. Fluorescence Microscopy

3.1. Surface Fluorescence

The hair fibers randomly collected were soaked in a solution of Rhodamine B (8 μg ml-1) for 20 minutes, followed by rinsing with deionized water for 1 minute, and dried at 45° C. for 15 minutes.

The fluorescence optical microscopy analysis was performed for the strands arranged longitudinally on a slide glass for microscopy using the Olympus BX53 microscope with the use of U-FGW filters.

For each study group, 30 fluorescence images of longitudinal segments were captured.

3.2. Cross-Sectional Fluorescence

Hair strands of each lock were collected, randomly, 24 hours after application of the product. Subsequently, the hair fibers were embedded in an acrylic resin—Historesin™, Leica, Benhein, following the procedures of drying and curing the resin. Cross sections were cut to a thickness of 10 μm using a glass knife and Ultramicrotome (Reichert-Jung, Heidelberg, Germany), followed by immersion in a solution of Rhodamine B (8 μg ml⁻¹). The slides were examined using the Olympus BX53 fluorescence microscope, using U-FGW filters.

From the images obtained, fluorescence intensities of 30 strands of hair were measured per treatment.

### 4. Software for Obtaining Average Values and Data Analysis:

CellSens® 1.4 (Olympus Corporation, Japan);

Scion Image for Windows (Scion, Corp, NIH, EUA);

GraphPad Prism 5.0 (GraphPad, California, EUA).

5. Results and Discussions

5.1. Microscopy of Longitudinal Segments—Fluorescence of Capillary Surface.

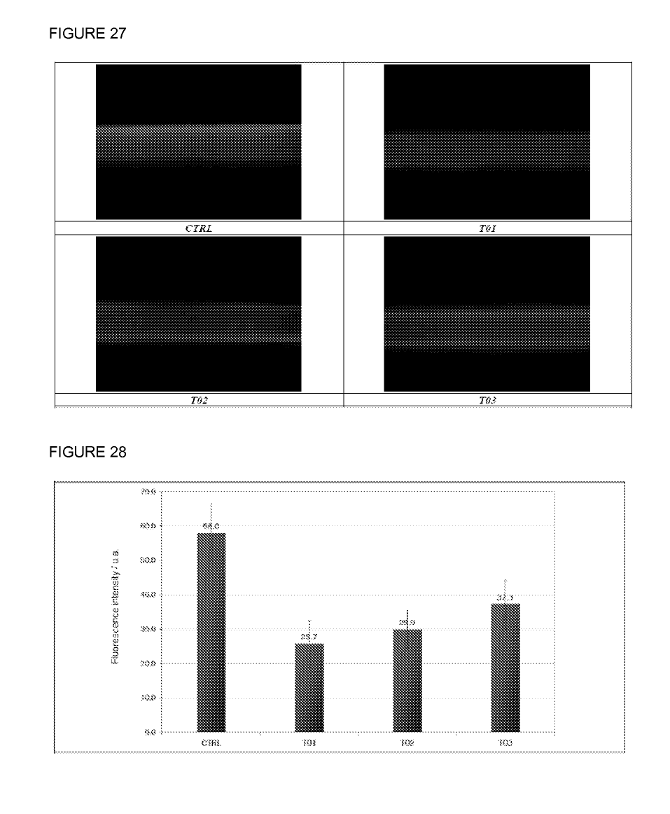

FIG. 27 illustrates the result of the evaluation by means of fluorescence microscopy of the longitudinal segments of hair fibers subjected to the treatment groups.

FIG. 28 illustrates the mean fluorescence intensities observed in the longitudinal segments of the fibers. The values for the mean fluorescence intensity are shown in Table 35:

TABLE 35
Data Obtained through the Image Analysis Software:
Scion ® for Windows Scion ® for Windows and whose
Fluorescence Intensity (a.u.) values were obtained from Scion ®
software for Windows.

| | | | |
|---|---|---|---|
| CTRL | T01 | T02 | T03 |

Surface Fluorescence

| | | | |
|---|---|---|---|
| 62.88 | 32.49 | 21.57 | 31.50 |
| 66.40 | 30.22 | 35.38 | 38.41 |
| 42.18 | 19.25 | 34.66 | 31.14 |
| 61.53 | 21.06 | 32.72 | 30.01 |
| 41.83 | 29.12 | 33.25 | 31.64 |
| 67.01 | 18.77 | 30.75 | 50.66 |
| 62.86 | 34.20 | 21.68 | 31.92 |
| 66.75 | 25.66 | 24.08 | 31.48 |
| 58.07 | 22.52 | 36.15 | 28.50 |
| 67.04 | 33.65 | 23.68 | 48.62 |
| 57.20 | 26.41 | 22.93 | 45.32 |
| 49.18 | 16.52 | 23.13 | 48.37 |
| 67.55 | 34.12 | 34.38 | 29.27 |
| 59.32 | 15.83 | 31.32 | 33.26 |
| 60.43 | 18.43 | 34.95 | 36.77 |
| 49.05 | 20.37 | 34.08 | 49.35 |
| 67.55 | 33.99 | 28.77 | 37.35 |
| 67.43 | 16.71 | 29.40 | 39.30 |
| 46.69 | 25.90 | 29.20 | 43.21 |
| 51.90 | 21.31 | 35.70 | 31.66 |
| 45.23 | 17.57 | 35.94 | 49.81 |
| 57.49 | 19.36 | 21.66 | 40.27 |
| 59.75 | 30.89 | 36.17 | 30.82 |
| 55.90 | 36.06 | 34.17 | 43.46 |
| 64.23 | 20.17 | 21.51 | 39.37 |
| 46.77 | 35.65 | 21.48 | 33.23 |
| 61.71 | 34.09 | 36.27 | 33.05 |
| 63.89 | 27.17 | 25.68 | 35.73 |
| 65.49 | 31.20 | 29.78 | 32.82 |
| 45.21 | 23.46 | 36.23 | 31.58 |

Cortical Fluorescence

| | | | |
|---|---|---|---|
| 38.21 | 14.62 | 14.06 | 21.57 |
| 41.45 | 14.83 | 15.47 | 22.92 |
| 44.64 | 13.15 | 12.80 | 20.36 |
| 41.20 | 11.38 | 14.34 | 18.41 |
| 45.24 | 10.55 | 13.99 | 19.03 |
| 37.25 | 14.78 | 10.89 | 18.51 |
| 39.09 | 8.89 | 14.00 | 20.38 |
| 33.52 | 13.73 | 13.97 | 19.62 |
| 35.51 | 9.01 | 13.36 | 21.43 |
| 38.47 | 8.84 | 12.71 | 20.10 |
| 45.08 | 9.07 | 14.65 | 21.11 |
| 43.15 | 11.01 | 16.46 | 18.87 |
| 38.32 | 15.24 | 15.79 | 19.84 |
| 38.74 | 13.72 | 15.80 | 20.42 |
| 44.20 | 13.11 | 13.25 | 18.68 |
| 33.36 | 12.56 | 12.54 | 22.86 |
| 32.38 | 9.37 | 12.85 | 22.63 |
| 42.94 | 10.68 | 11.79 | 20.56 |
| 41.19 | 11.21 | 10.48 | 18.63 |
| 40.29 | 12.84 | 14.74 | 19.82 |
| 40.96 | 10.28 | 15.28 | 19.69 |
| 42.45 | 9.68 | 15.65 | 18.99 |
| 33.95 | 11.61 | 15.01 | 21.07 |
| 41.34 | 9.51 | 16.87 | 22.62 |
| 33.21 | 8.40 | 14.71 | 20.48 |
| 38.76 | 10.49 | 11.95 | 22.41 |
| 38.60 | 11.77 | 11.11 | 21.68 |
| 42.38 | 15.14 | 16.04 | 17.26 |
| 34.30 | 8.26 | 15.66 | 18.96 |
| 41.05 | 15.54 | 15.59 | 18.52 |

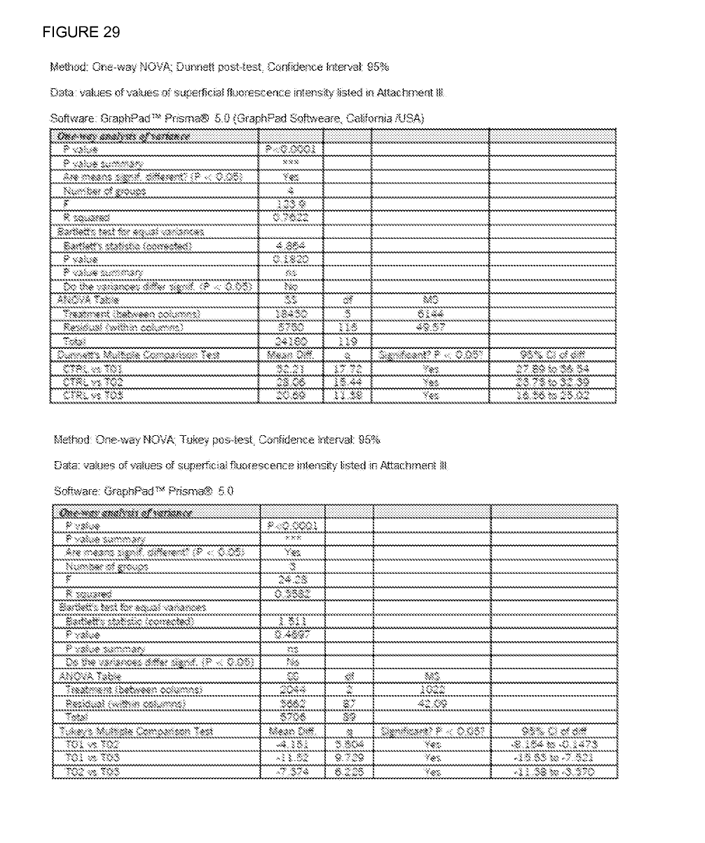

The surface fluorescence intensity data in the study groups were statistically compared with the CTRL group using the method of analysis of single factor variance, with Dunnett multiple comparison post-test, considering a confidence interval of 95%. Table 36 shows the results of statistical analysis. The complete statistical analysis is described in FIG. 29.

TABLE 36
Results of statistical analysis. P values for I.C. of 95%
Comparison between treatments

| Comparison | P value | Did it show significant differences? |
|---|---|---|
| CTRL vs T01 | P < 0.05 | Yes |
| CTRL vs T02 | P < 0.05 | Yes |
| CTRL vs T03 | P < 0.05 | Yes |

According to the results obtained, the locks subjected to treatment T01, T02 and T03 exhibited significantly lower fluorescence surface intensity in relation to the locks subjected to CTRL treatment.

The study groups were statistically compared with each other using the method of single-factor analysis of variance, with Tukey multiple comparison post-test, considering a confidence interval of 95%. Table 37 shows the results of statistical analysis. The complete statistical analysis is described in FIG. 29.

TABLE 37
Results of statistical analysis. P values for I.C. of 95% Comparison
between treatments

| Comparison | P value | Did it show significant differences? |
|---|---|---|
| T01 vs T02 | P < 0.05 | Yes |
| T01 vs T03 | P < 0.05 | Yes |
| T02 vs T03 | P < 0.05 | Yes |

According to the results obtained, the locks subjected to treatment T01 exhibited significantly lower fluorescence surface intensity when compared to the locks subjected to treatments T02 and T03.

The locks subjected to treatment T02 exhibited significantly lower fluorescence surface intensity when compared to the locks subjected to treatment T03.

Using equations 1 and 2, we calculated the “Reduction of Fluorescence Intensity RI” of the treatment groups in relation to the CTRL treatment, in percentage or number of times, respectively. The results obtained are listed in Table 38.

$$RI \% = 100 * ( \frac{I {\%}_{CTRL} - I {\%}_{TRAT}}{I {\%}_{CTRL}} )$$

Equation 1: Calculation of Reduced Fluorescence Intensity (%), where I=fluorescence intensity. TRAT=Fluorescence intensity values for the treatments. CTRL=Fluorescence intensity values for the CTRL group.

$$RI = \frac{I {\%}_{CTRL}}{I {\%}_{TRAT}}$$

Equation 2: Calculation of Reduced Fluorescence Intensity (number of times), where I=fluorescence intensity. TRAT=Fluorescence intensity values for the treatments. CTRL=Fluorescence intensity values for the CTRL group.

TABLE 38
Reduction of Surface Fluorescence Intensity (percentage and
number of times) of the study groups in relation to the CTRL group

| Treatment | % | Number of times |
|---|---|---|
| T01 | 56 | 2.3 |
| T02 | 48 | 1.9 |
| T03 | 36 | 1.6 |

5.2. Cross-Sectional Microscopy-Cortical Fluorescence

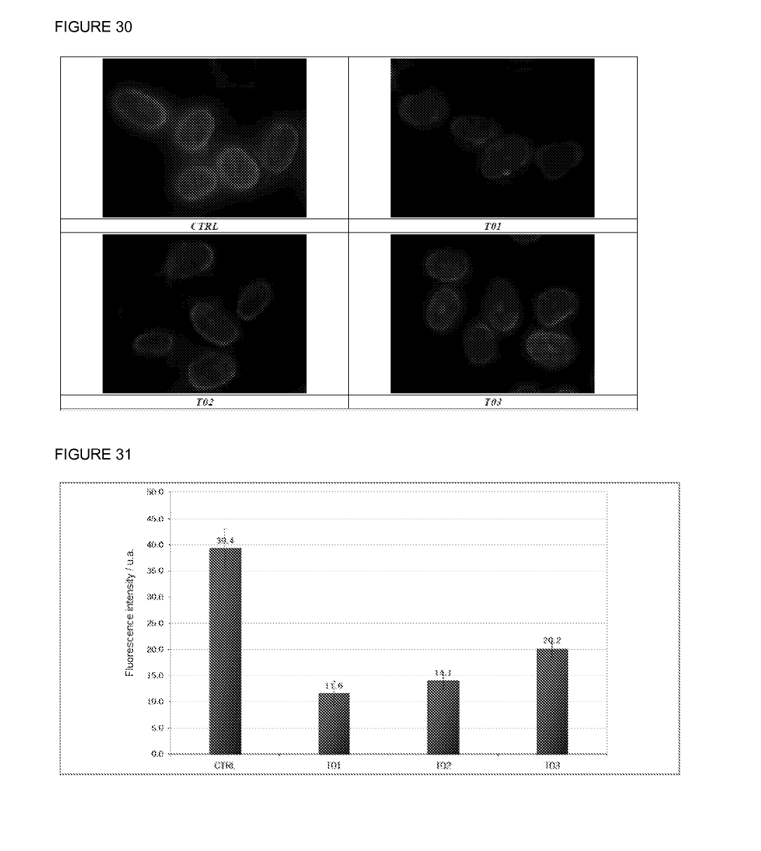

FIG. 30 illustrates the result of the evaluation by means of fluorescence microscopy of the cross sections of hair fibers subjected to the treatment groups.

FIG. 31 illustrates the mean fluorescence intensities observed in the cross sections of the fibers. The values for the mean fluorescence intensity are shown in Table 35 above:

The cortical fluorescence intensity data in the study groups were statistically compared with the CTRL group using the method of analysis of single factor variance, with Dunnett multiple comparison post-test, considering a confidence interval of 95%. Table 39 shows the results of statistical analysis. The complete statistical analysis is described in FIG. 29.

TABLE 39
Results of statistical analysis. P values for I.C. of 95% Comparison
between treatments

| | | Did it show significant |
|---|---|---|
| Comparison | P value | differences? |
| CTRL vs T01 | P < 0.05 | Yes |
| CTRL vs T02 | P < 0.05 | Yes |
| CTRL vs T03 | P < 0.05 | Yes |

According to the results obtained, the locks subjected to treatment T01, T02 and T03 exhibited significantly lower fluorescence surface intensity in relation to the locks subjected to CTRL treatment.

The study groups were statistically compared with each other using the method of single-factor analysis of variance, with Tukey multiple comparison post-test, considering a confidence interval of 95%. Table 40 shows the results of statistical analysis. The complete statistical analysis is described in FIG. 29.

TABLE 40
Results of statistical analysis. P values for I.C. of 95% Comparison
between treatments

| | | Did it show significant |
|---|---|---|
| Comparison | P value | differences? |
| T01 vs T02 | P < 0.05 | Yes |
| T01 vs T03 | P < 0.05 | Yes |
| T02 vs T03 | P < 0.05 | Yes |

According to the results obtained, the locks subjected to treatment T01 exhibited significantly lower cortical fluorescence intensity when compared to the locks subjected to treatments T02 and T03.

The locks subjected to treatment T02 exhibited significantly lower cortical fluorescence intensity when compared to the locks subjected to treatment T03.

Using equations 1 and 2, we calculated the “Reduction of Fluorescence Intensity RI” of the treatment groups in relation to the CTRL treatment, in percentage or number of times, respectively. The results obtained are described in Table 41.

TABLE 41
Reduction of Surface Fluorescence Intensity (percentage and
number of times) of the study groups in relation to the CTRL group.

| Treatment | % | Number of times |
|---|---|---|
| T01 | 70 | 3.4 |
| T02 | 64 | 2.8 |
| T03 | 49 | 1.9 |

### 6. Conclusion

In the present study, the fluorescent microscopy technique was used to evaluate the adsorption of the active ingredients in the capillary surface and the penetration of active ingredients into the hair cortex. Longitudinal and cross sectional cuts of hair were immersed in a fluorescent marker dye, Rhodamine B, so as to mark damaged hair sites. The greater the fluorescence intensity, the greater the amount of dye bound to damaged sites.

When a high substantiveness product is applied to the hair, there is a link between the active ingredients and the damaged hair sites, thus the number of sites available for binding to the marker dye is reduced. Consequently, the fluorescence intensity is lower.

In the present study, locks of bleached caucasian hair were subjected to treatments with the following product:
SLES 10% —CTRL group;
Mixture 1—group T01;
Mixture 2—group T02;
Mixture 3—group T03;

According to the results obtained, the locks subjected to treatments T01, T02 and T03 exhibited significantly lower fluorescence surface intensity values in relation to the locks subjected to CTRL treatment.

The locks subjected to treatment T01 exhibited significantly lower surface and cortical fluorescence intensity values when compared to the locks subjected to treatments T02 and T03.

The locks subjected to treatment T02 exhibited significantly lower surface and cortical fluorescence intensity values when compared to the locks subjected to treatment T03.

TABLE 42
Reduction of Surface and Cortical Fluorescence Intensity
(percentage and number of times) of the study groups in relation to the
CTRL group

| | |
|---|---|
| Surface | Cortical |

| | | Number of | | Number of |
|---|---|---|---|---|
| Treatment | % | times | % | times |
| T01 | 70 | 3.4 | 56 | 2.3 |
| T02 | 64 | 2.8 | 48 | 1.9 |
| T03 | 49 | 1.9 | 36 | 1.6 |

1. A composition for cosmetic formulation, characterized by consisting of a mixture of up to 3 components selected from murumuru butter, ucuuba butter, Brazilian-nut oil, passion fruit oil, cupuassu butter, assai oil and nhandiroba oil and/or esters thereof, and/or esters thereof.

2. The composition for cosmetic composition according to claim 1, characterized in that the ester may be myristyl cupuassuate.

3. The composition for cosmetic formulation according to claim 1, characterized by comprising from 0 to 40% murumuru butter, from 0 to 40% ucuuba butter and from 0 to 60% Brazilian-nut oil.

4. The composition for cosmetic formulation according to claim 3, characterized by comprising:
25% murumuru butter;
25% ucuuba butter; and
50% Brazilian-nut oil.

5. The composition for cosmetic formulation according to claim 1, characterized by comprising from 0 to 70% passion fruit oil, from 0 to 50% cupuassu butter, and from 0 to 30% myristyl cupuassuate.

6. The composition for cosmetic formulation according to claim 5, characterized by comprising:
50% passion fruit oil;
35% cupuassu butter; and
15% myristyl cupuassuate.

7. The composition for cosmetic formulation according to claim 1, characterized by comprising from 1 to 30% assai oil and from 70 to 99% nhandiroba oil.

8. The composition for cosmetic formulation according to claim 7, characterized by comprising:
10% assai oil; and
90% nhandiroba oil.

9. Use of a composition as defined in claim 1, characterized by being for the preparation of cosmetic products.

10. A cosmetic product, characterized by comprising a composition as defined in claim 1.
